(19) United States
(12) Patent Application Publication
(10) Pub. No.: US 2024/0207645 A1
(43) Pub. Date: Jun. 27, 2024

(54) RADIOTHERAPY OPTIMIZATION FOR ARC SEQUENCING AND APERTURE REFINEMENT

(71) Applicant: Elekta, Inc., Atlanta, GA (US)

(72) Inventor: Lyndon Stanley Hibbard, St. Louis, MO (US)

(21) Appl. No.: 18/567,677

(22) PCT Filed: Jun. 24, 2021
(86) PCT No.: PCT/US2021/070766

§ 371 Date: Dec. 6, 2023

(51) Int. Cl. A61N 5/10; G05B 13/02

(52) U.S. Cl. CPC A61N 5/1039; A61N 5/1045; A61N 5/1081; G05B 13/027

(57) ABSTRACT

Systems and methods are disclosed for generating radiotherapy machine parameters used in a radiotherapy treatment plan, based on machine learning prediction. The systems and methods include: obtaining three-dimensional image data which indicates target dose areas and organs-at-risk areas of a subject; generating anatomy projection images from the image data, each anatomy projection image providing a view from a respective beam angle of the radiotherapy treatment; using a trained neural network model (trained with corresponding pairs of anatomy projection images and control point images) to generate control point images, each control point image indicating an intensity and aperture(s) of a control point of the radiotherapy treatment to apply at a respective beam angle; and generating a set of final control points for use in the radiotherapy treatment to control a radiotherapy treatment machine, based on optimization of the control points indicated by the generated control point images.

### TECHNICAL FIELD

Embodiments of the present disclosure pertain generally to determining plan parameters that direct the radiation therapy performed by a radiation therapy treatment system. In particular, the present disclosure pertains to using machine learning technologies to determine arc sequencing and aperture values of control points, used in a treatment plan for a radiation therapy system.

### BACKGROUND

Radiation therapy (or “radiotherapy”) can be used to treat cancers or other ailments in mammalian (e.g., human and animal) tissue. One such radiotherapy technique is provided using a Gamma Knife, by which a patient is irradiated by a large number of low-intensity gamma rays that converge with high intensity and high precision at a target (e.g., a tumor). Another such radiotherapy technique is provided using a linear accelerator (linac), whereby a tumor is irradiated by high-energy particles (e.g., electrons, protons, ions, high-energy photons, and the like). The placement and dose of the radiation beam must be accurately controlled to ensure the tumor receives the prescribed radiation, and the placement of the beam should be such as to minimize damage to the surrounding healthy tissue, often called the organ(s) at risk (OARs). Radiation is termed “prescribed” because a physician orders a predefined amount of radiation to the tumor and surrounding organs similar to a prescription for medicine. Generally, ionizing radiation in the form of a collimated beam is directed from an external radiation source toward a patient.

A specified or selectable beam energy can be used, such as for delivering a diagnostic energy level range or a therapeutic energy level range. Modulation of a radiation beam can be provided by one or more attenuators or collimators (e.g., a multi-leaf collimator (MLC)). The intensity and shape of the radiation beam can be adjusted by collimation to avoid damaging healthy tissue (e.g., OARs) adjacent to the targeted tissue by conforming the projected beam to a profile of the targeted tissue.

The treatment planning procedure may include using a three-dimensional (3D) image of the patient to identify a target region (e.g., the tumor) and to identify critical organs near the tumor. Creation of a treatment plan can be a time-consuming process where a planner tries to comply with various treatment objectives or constraints (e.g., dose volume histogram (DVH), overlap volume histogram (OVH)), taking into account their individual importance (e.g., weighting) in order to produce a treatment plan that is clinically acceptable. This task can be a time-consuming trial-and-error process that is complicated by the various OARs because as the number of OARs increases (e.g., a dozen or more OARs for a head-and-neck treatment), so does the complexity of the process. OARs distant from a tumor may be easily spared from radiation, while OARs close to or overlapping a target tumor may be difficult to spare.

Traditionally, for each patient, the initial treatment plan can be generated in an “offline” manner. The treatment plan can be developed well before radiation therapy is delivered, such as using one or more medical imaging techniques. Imaging information can include, for example, images from X-rays, computed tomography (CT), nuclear magnetic resonance (MR), positron emission tomography (PET), single-photon emission computed tomography (SPECT), or ultrasound. A health care provider, such as a physician, may use 3D imaging information indicative of the patient anatomy to identify one or more target tumors along with the OARs near the tumor(s). The health care provider can delineate the target tumor that is to receive a prescribed radiation dose using a manual technique, and the health care provider can similarly delineate nearby tissue, such as organs, at risk of damage from the radiation treatment. Alternatively or additionally, an automated tool (e.g., ABAS provided by Elekta AB, Sweden) can be used to assist in identifying or delineating the target tumor and organs at risk. A radiation therapy treatment plan (“treatment plan”) can then be created using numerical optimization techniques the minimize objective functions composed of clinical and dosimetric objectives and constraints (e.g., the maximum, minimum, and fraction of dose of radiation to a fraction of the tumor volume (“95% of target shall receive no less than 100% of prescribed dose”), and like measures for the critical organs). The optimized plan is comprised of numerical parameters that specify the direction, cross-sectional shape, and intensity of each radiation beam.

The treatment plan can then be later executed by positioning the patient in the treatment machine and delivering the prescribed radiation therapy directed by the optimized plan parameters. The radiation therapy treatment plan can include dose “fractioning,” whereby a sequence of radiation treatments is provided over a predetermined period of time (e.g., 30-45 daily fractions), with each treatment including a specified fraction of a total prescribed dose.

As part of the treatment planning process for radiotherapy dosing, fluence is also determined and evaluated, followed by a translation of such fluence into control points for delivering dosage with a radiotherapy machine. Fluence is the density of radiation photons or particles normal to the beam direction, whereas dose is related to the energy released in the material when the photons or particles interact with the material atoms. Dose is therefore dependent on the fluence and the physics of the radiation-matter interactions. Significant planning is conducted as part of determining fluence, dosing, and dosing delivery for a particular patient and treatment plan.

### OVERVIEW

In some embodiments, methods, systems and computer-readable medium are provided for generating radiotherapy machine parameters (such as control point apertures) used as part of one or more radiotherapy treatment plans. The methods, systems and computer-readable medium perform operations comprising: obtaining a three-dimensional set of image data corresponding to a subject for radiotherapy treatment, the image data indicating one or more target dose areas and one or more organs-at-risk areas in anatomy of the subject; generating anatomy projection images from the image data, each anatomy projection image providing a view of the subject from a respective beam angle of the radiotherapy treatment; using a trained neural network model to generate control point images based on the anatomy projection images, each of the control point images indicating an intensity and one or more apertures of a control point of the radiotherapy treatment to apply at a respective beam angle, where the neural network model is trained with corresponding pairs of training anatomy projection images and training control point images; and generating a set of final control points for use in the radiotherapy treatment to control a radiotherapy treatment machine, based on optimization of the control points of the radiotherapy treatment indicated by the generated control point images.

In an example, the beam angles of the radiotherapy treatment correspond to gantry angles of the radiotherapy treatment machine, and obtaining the three-dimensional set of image data corresponding to a subject includes obtaining image data for each gantry angle of the radiotherapy treatment machine. In such a scenario, each generated anatomy projection image represents a view of the anatomy of the subject from a given gantry angle used to provide treatment with a given radiotherapy beam.

In an example, each anatomy projection image is generated by forward projection of the three-dimensional set of image data at respective angles of multiple beam angles. Also in an example, optimization of the control points produces a pareto-optimal plan used in the radiotherapy treatment plan for the subject.

In an example, the radiotherapy treatment comprises a volume modulated arc therapy (VMAT) radiotherapy performed by the radiotherapy treatment machine, and multiple radiotherapy beams are shaped to achieve a modulated dose for target areas, from among multiple beam angles, to deliver a prescribed radiation dose.

In an example, the optimization of the control points includes performing direct aperture optimization with aperture settings, with the set of final control points includes control points corresponding to each of multiple radiotherapy beams. In this scenario, performing the radiotherapy treatment includes using the set of final control points, with the set of final control points being used to control multi-leaf collimator (MLC) leaf positions of a radiotherapy treatment machine at a given gantry angle corresponding to a given beam angle.

In an example, the operations also include using fluence data to determine radiation doses in the radiotherapy treatment plan, with the trained neural network model being further configured to generate the control point images based on the fluence data. For instance, the fluence data may be provided from fluence maps, and the neural network model may be further trained with fluence maps corresponding to the training anatomy projection images and the training control point images. Additionally, the fluence maps may be provided from use of a second trained neural network model that is configured to generate the fluence maps based on the anatomy projection images, each of the generated fluence maps indicating a fluence distribution of the radiotherapy treatment at a respective beam angle, as the second neural network model is trained with corresponding pairs of the anatomy projection images and fluence maps.

In an example, training of the neural network model uses pairs of anatomy projection images and control point images for a plurality of human subjects, with each individual pair being provided from a same human subject. Such training of the neural network model may include: obtaining multiple sets of training anatomy projection images, each set of the training anatomy projection images indicating one or more target dose areas and one or more organs-at-risk areas in the anatomy of a respective subject; obtaining multiple sets of training control point images corresponding to the training anatomy projection images, each set of the training control point images indicating a control point for at a respective beam angle of the radiotherapy machine used with radiotherapy treatment of the respective subject; and training the neural network model based on the training anatomy projection images that correspond to the training control point images.

In some examples, the trained neural network model is a generative model of a generative adversarial network (GAN) comprising at least one generative model and at least one discriminative model, where the at least one generative model and the at least one discriminative model correspond to respective generative and discriminative convolutional neural networks. In some examples, this GAN comprises a conditional generative adversarial network (cGAN).

The above overview is intended to provide an overview of subject matter of the present patent application. It is not intended to provide an exclusive or exhaustive explanation of the inventive subject matter. The detailed description is included to provide further information about the present patent application.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates an exemplary radiotherapy system, according to some examples.

FIGS. 2A and 2B illustrate projection views of an ellipse and an exemplary prostate target anatomy, according to some examples.

FIG. 3A illustrates an exemplary radiation therapy system that can include radiation therapy output configured to provide a therapy beam, according to some examples.

FIG. 3B illustrates an exemplary system including a combined radiation therapy system and an imaging system, such as a cone beam computed tomography (CBCT) imaging system, according to some examples.

FIG. 4 illustrates a partially cut-away view of an exemplary system including a combined radiation therapy system and an imaging system, such as a nuclear magnetic resonance (MR) imaging (MRI) system, according to some examples.

FIG. 5 illustrates an exemplary Gamma Knife radiation therapy system, according to some examples.

FIGS. 6A and 6B depict the differences between an exemplary MRI image and a corresponding CT image, respectively, according to some examples.

FIG. 7 illustrates an exemplary collimator configuration for shaping, directing, or modulating an intensity of a radiation therapy beam, according to some examples.

FIG. 8 illustrates a data flow and processes for radiotherapy plan development, according to some examples.

FIG. 9 illustrates an example of control point aperture calculation operations, according to some examples.

FIG. 10 illustrates an example of anatomical projections and radiotherapy treatment constraints at multiple angles of a radiotherapy treatment, according to some examples.

FIG. 11 illustrates example transformations of images and control point parameters into 3D image volumes, according to some examples.

FIG. 12 illustrates an example of control point apertures at multiple angles of a radiotherapy treatment, according to some examples.

FIG. 13 illustrates a deep learning procedure to train a model to predict control point parameters from projection image data and control point parameter data, according to some examples.

FIG. 14 illustrates results of a training procedure to generate control point parameters in various types of neural network models, according to some examples.

FIGS. 15A and 15B respectively depict a schematic of generative and discriminative deep convolutional neural networks used in generating and discriminating control point representations, according to some examples.

FIG. 16 depicts schematics of a generative adversarial network used for training a generative model for predicting control point representations, according to some examples.

FIGS. 17 and 18 illustrate respective data flows for training and use of a machine learning model adapted to produce simulated control point representations, according to some examples.

FIG. 19 illustrates a method for generating control points used in a radiotherapy treatment plan and generating the machine parameters to deliver the radiotherapy treatment plan according to the control points, according to some examples.

FIG. 20 illustrates an exemplary block diagram of a machine on which one or more of the methods as discussed herein can be implemented.

### DETAILED DESCRIPTION

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized and that structural, logical and electrical changes may be made without departing from the scope of the present disclosure. The following detailed description is, therefore, not to be taken in a limiting sense, and the scope of the present disclosure is defined by the appended claims and their equivalents.

Intensity modulated radiotherapy (IMRT) and volumetric modulated arc therapy (VMAT) have become the standards of care in modern cancer radiation therapy. Creating individual patient IMRT or VMAT treatment plans is often a trial-and-error process, weighing target dose versus OAR sparing tradeoffs, and adjusting program constraints whose effects on the plan quality metrics and the dose distribution can be very difficult to anticipate. Indeed, the order in which the planning constraints are adjusted can itself result in dose differences. Treatment plan quality depends on often subjective judgements by the planner that depend on his/her experience and skill. Even the most skilled planners still have no assurance that their plans are close to the best possible, or whether a little or a lot of effort will result in a significantly better plan.

The present disclosure includes various techniques to improve and enhance radiotherapy treatment by generating control point values for use within IMRT or VMAT treatment, with use of a model-enhanced process for assisting radiotherapy plan design. This model may comprise a trained machine learning model, such as an artificial neural network model, which is trained to produce (predict) a computer-modeled, image-based representation of control point values from a given input. These control point values may be subsequently used for implementing radiotherapy treatment machine parameters, with the control points being used to control radiotherapy machine operations that deliver radiation therapy with treatment to a patient's delineated anatomy.

The technical benefits of these techniques include reduced radiotherapy treatment plan creation time, improved quality in generated radiotherapy treatment plans, and the evaluation of less data or user inputs to produce higher quality control point designs and radiotherapy machine treatment plans. Such technical benefits may result in many apparent medical treatment benefits, including improved accuracy of radiotherapy treatment, reduced exposure to unintended radiation, and the like. The disclosed techniques may be applicable to a variety of medical treatment and diagnostic settings or radiotherapy treatment equipment and devices, including but not limited to the use of IMRT and VMAT treatment plans.

Development of IMRT and VMAT treatment plans is conventionally performed from the selection, adjustment, and optimization of control points, based on a 3D dose distribution covering the target while attempting to minimize the effect of dose on nearby OARs. Such a 3D dose distribution is often produced from a fluence map, as a fluence 3D dose distribution (often represented with a fluence map) is resampled and transformed to accommodate linac and multileaf collimator (MLC) properties to become a clinical, deliverable treatment plan. This fluence is translated into appropriately weighted beamlets that can be directed through the linac MLC from many angles around the target, to achieve the desired dose in tissues itself. VMAT radiotherapy may have 100 or more beams with the total numbers of beamlet weights equal to 10⁵ or more.

Among other techniques, the following discusses creation and training of an anatomy-dependent model of radiotherapy doses so that a resulting set of control points can be identified closer to a set of ideal end values. Such a model may accept as input a combination of input patient images and OAR data, to output control point values. Such control point values may be used as part of arc sequencing and aperture optimization. Additionally, such an anatomy-dependent model of the radiotherapy may be adapted for verification or validation of control point values, and integrated in a variety of ways for radiotherapy planning.

In an example, this anatomy-dependent model is implemented with a machine learning method to predict treatment plan parameters that can serve an aid to shorten the computational time for conventional VMAT arc sequencing and aperture optimization. By deriving predictions from a model of clinical plans, the predictions can produce a higher quality of plan than default commercial algorithms that do not account for differences between incoming new patients. Among other benefits, such that machine learning predictions of patient plans can be used to shorten the time to produce clinically useful VMAT plans by reducing the time for arc sequencing and aperture refinement. Further, the machine learning predictions of patient plans will result in VMAT plans with higher quality than VMAT plans produced from commercial segmentation algorithms.

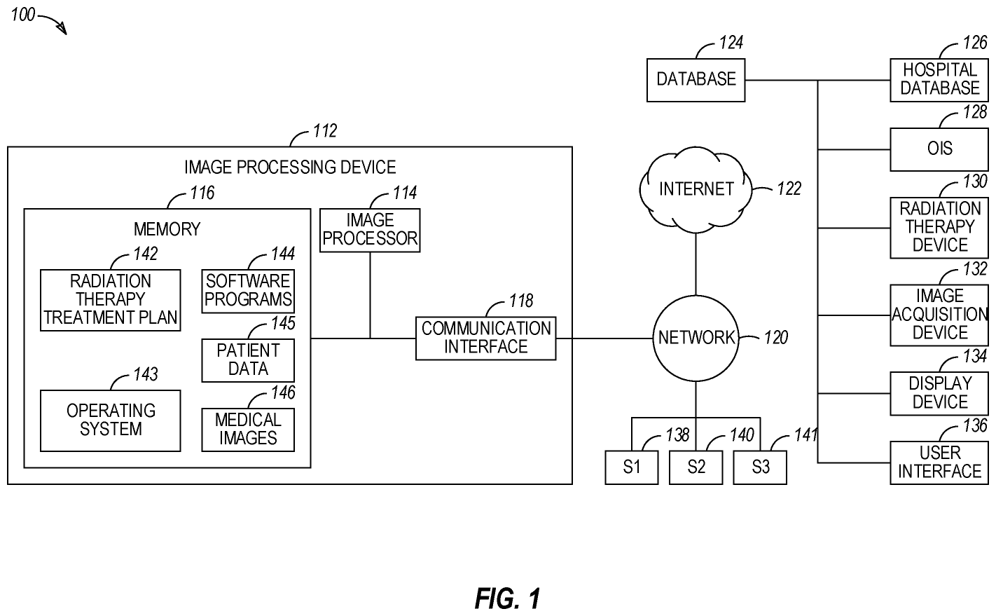

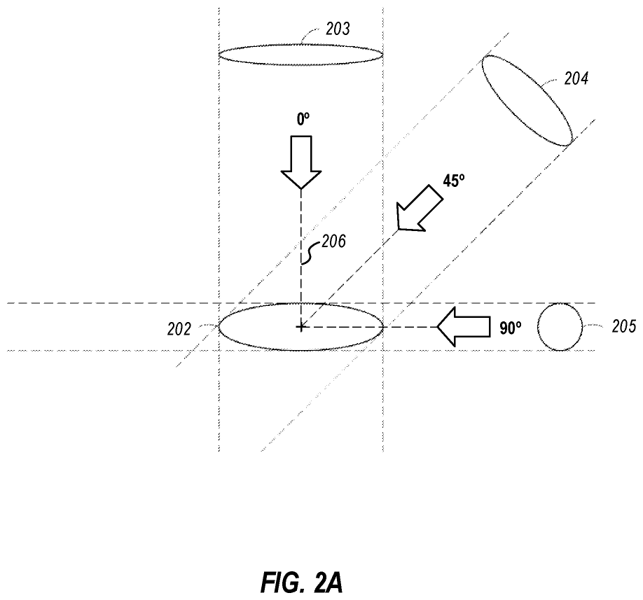

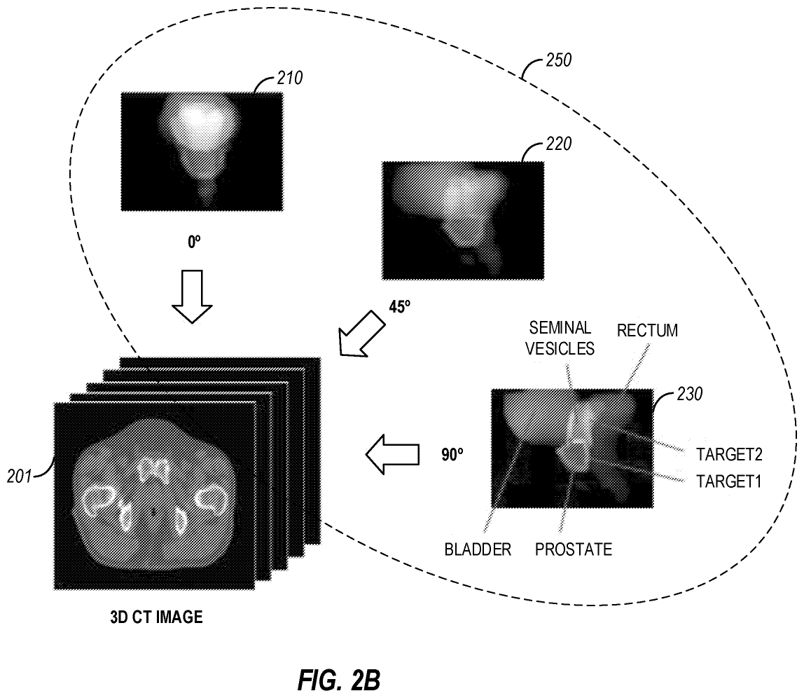

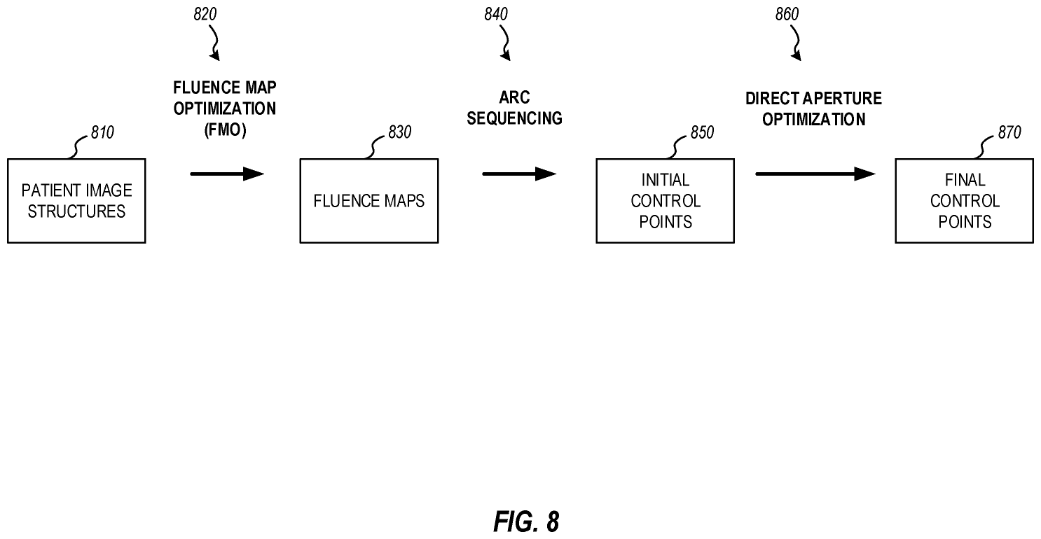

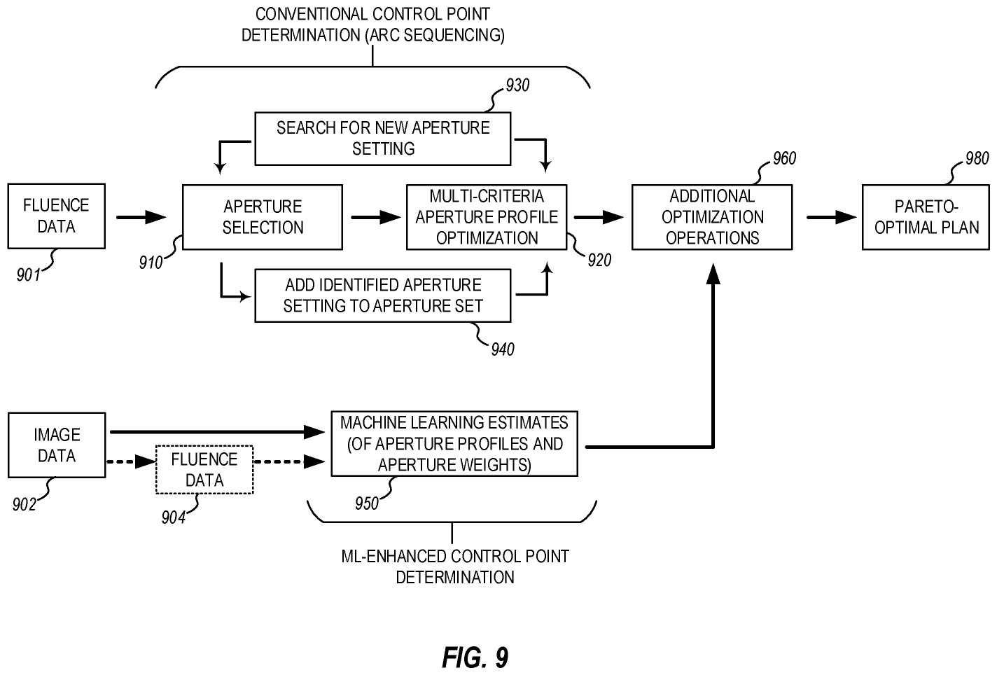

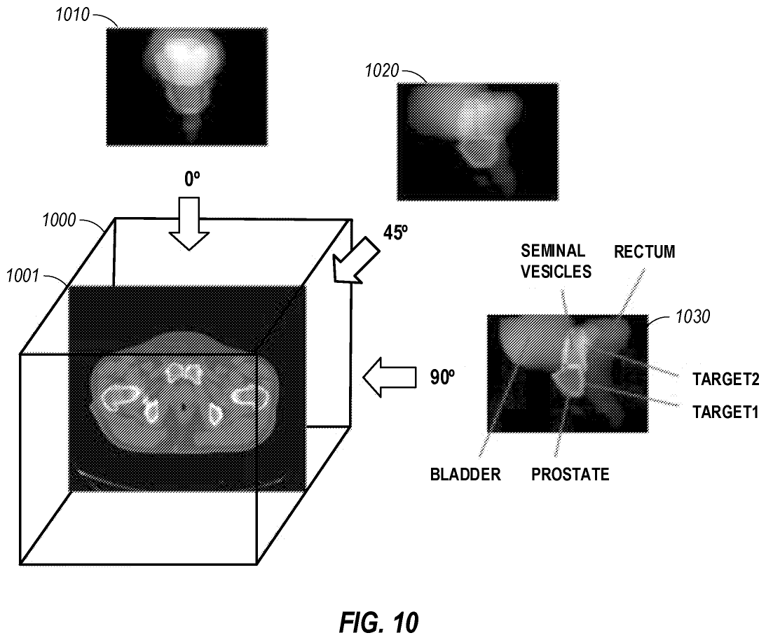

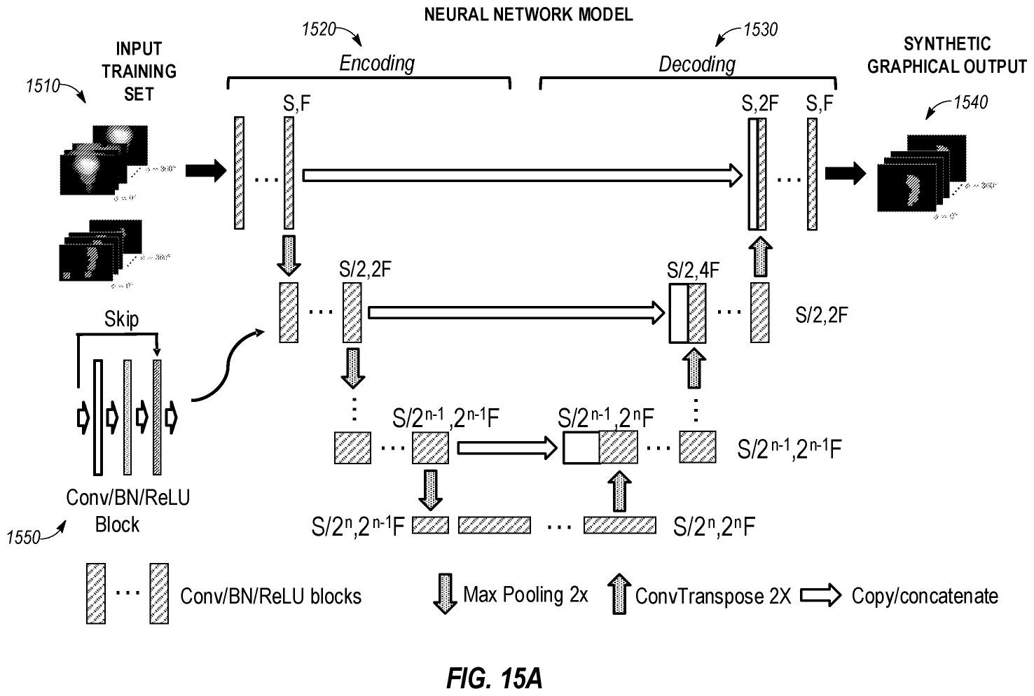

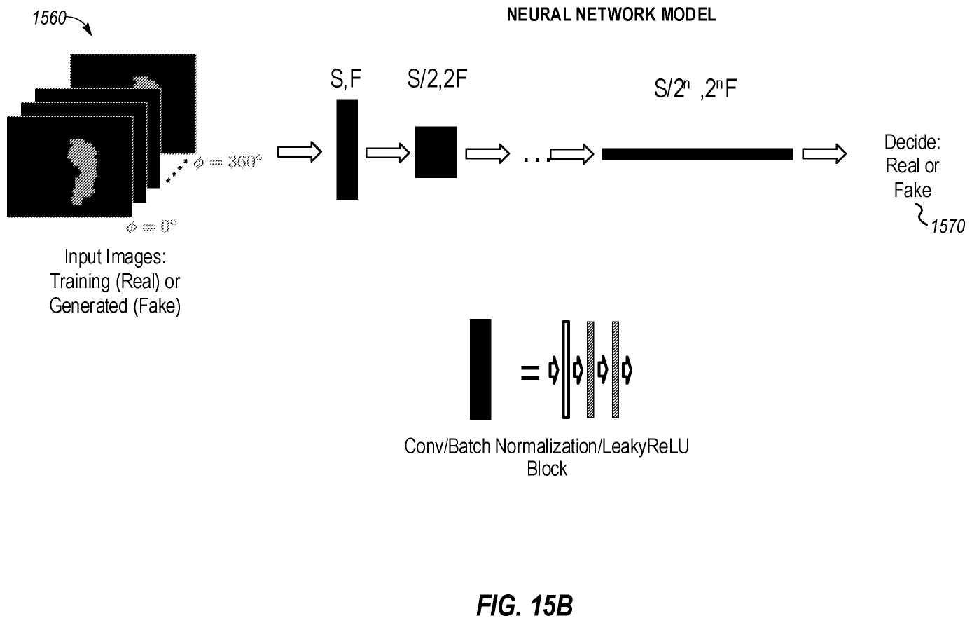

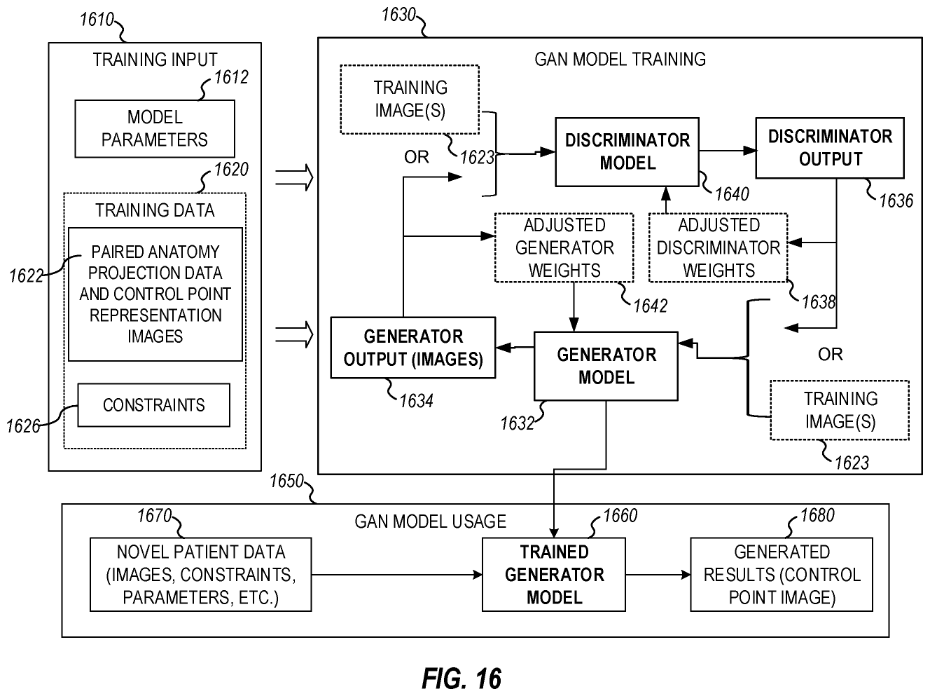

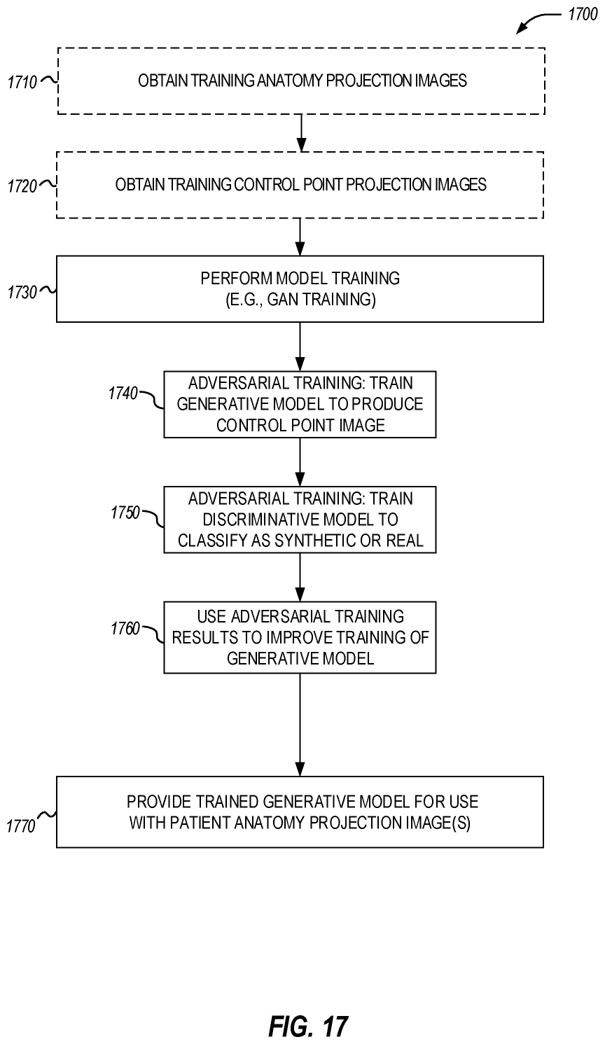

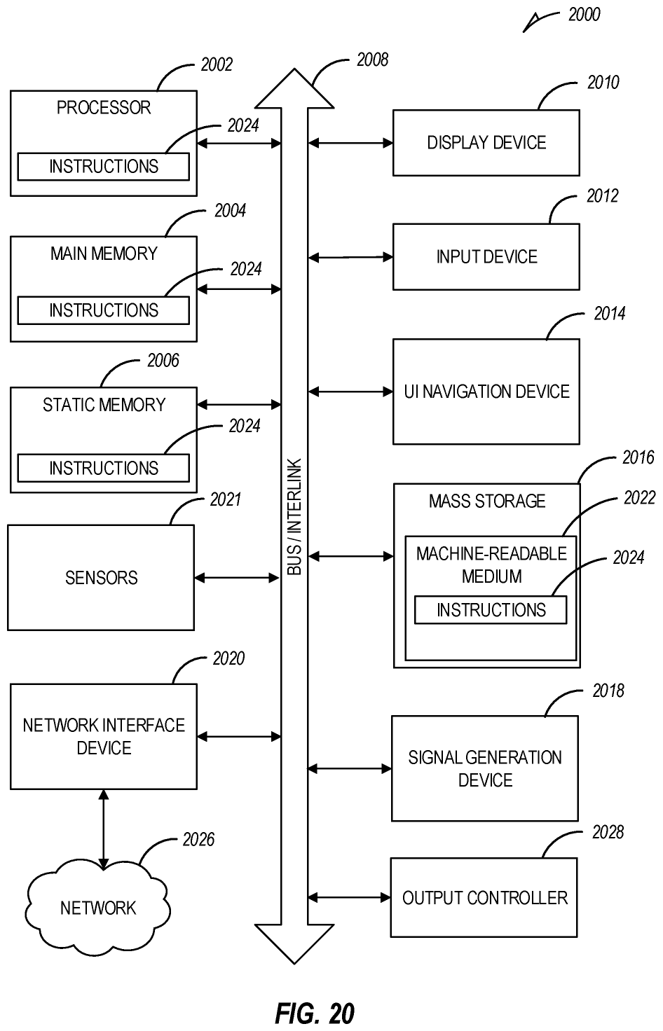

The following paragraphs provide an overview of example radiotherapy system implementations and treatment planning (with reference to FIGS. 2A to 7), including with the use of computing systems and hardware implementations (with reference to FIGS. 1 and 20). The following also provides a discussion of considerations specific to radiotherapy control point parameters (with reference to FIGS. 8 to 9) and generation of these control point parameters relative to patient anatomy projections (with reference to FIGS. 10 to 14). Finally, a discussion of machine learning techniques (with reference to FIGS. 15A to 16B) is provided for methods of training and using a machine learning model (FIGS. 17 to 19).

FIG. 1 illustrates a radiotherapy system 100 for providing radiation therapy to a patient. The radiotherapy system 100 includes an image processing device 112. The image processing device 112 may be connected to a network 120. The network 120 may be connected to the Internet 122. The network 120 can connect the image processing device 112 with one or more of a database 124, a hospital database 126, an oncology information system (OIS) 128, a radiation therapy device 130, an image acquisition device 132, a display device 134, and a user interface 136. The image processing device 112 can be configured to generate radiation therapy treatment plans 142 and plan-related data to be used by the radiation therapy device 130.

The image processing device 112 may include a memory device 116, an image processor 114, and a communication interface 118. The memory device 116 may store computer-executable instructions, such as an operating system 143, radiation therapy treatment plans 142 (e.g., original treatment plans, adapted treatment plans and the like), software programs 144 (e.g., executable implementations of artificial intelligence, deep learning neural networks, radiotherapy treatment plan software), and any other computer-executable instructions to be executed by the processor 114. In an example, the software programs 144 may convert medical images of one format (e.g., MRI) to another format (e.g., CT) by producing synthetic images, such as pseudo-CT images. For instance, the software programs 144 may include image processing programs to train a predictive model for converting a medical image 146 in one modality (e.g., an MRI image) into a synthetic image of a different modality (e.g., a pseudo CT image); alternatively, the image processing programs may convert a CT image into an MRI image. In another example, the software programs 144 may register the patient image (e.g., a CT image or an MR image) with that patient's dose distribution (also represented as an image) so that corresponding image voxels and dose voxels are associated appropriately by the network. In yet another example, the software programs 144 may substitute functions of the patient images such as signed distance functions or processed versions of the images that emphasize some aspect of the image information. Such functions might emphasize edges or differences in voxel textures, or any other structural aspect useful to neural network learning. In another example, the software programs 144 may substitute functions of a dose distribution that emphasizes some aspect of the dose information. Such functions might emphasize steep gradients around the target or any other structural aspect useful to neural network learning. The memory device 116 may store data, including medical images 146, patient data 145, and other data required to create and implement at least one radiation therapy treatment plan 142 or data associated with at least one plan.

In yet another example, the software programs 144 may generate projection images for a set of two-dimensional (2D) and/or 3D CT or MR images depicting an anatomy (e.g., one or more targets and one or more OARs) representing different views of the anatomy from one or more beam angles used to deliver radiotherapy, which may correspond to respective gantry angles of the radiotherapy equipment. For example, the software programs 144 may process the set of CT or MR images and create a stack of projection images depicting different views of the anatomy depicted in the CT or MR images from various perspectives of the radiotherapy beams, as part of generating control point apertures used in a radiotherapy treatment plan. For instance, one projection image may represent a view of the anatomy from 0 degrees of the gantry, a second projection image may represent a view of the anatomy from 45 degrees of the gantry, and a third projection image may represent a view of the anatomy from 90 degrees of the gantry, with a separate radiotherapy beam being located at each angle. In other examples, each projection image may represent a view of the anatomy from a particular beam angle, corresponding to the position of the radiotherapy beam at the respective angle of the gantry.

Projection views for a simple ellipse 202 are shown schematically in FIG. 2A. Here, the views are oriented relative to the ellipse center and capture the shape and extent of the ellipse 202 as seen from each angle (e.g., 0 degrees represented by view 203, 45 degrees represented by view 204, and 90 degrees represented by view 205). For example, the view of ellipse 202 when seen from a 0-degree angle relative to the y-axis 206 of ellipse 202 is projected as view 203. For example, the view of ellipse 202 when seen from a 45-degree angle relative to the y-axis 206 of ellipse 202 is projected as view 204. For example, the view of ellipse 202 when seen from a 90-degree angle relative to the y-axis 206 of ellipse 202 is projected as view 205.

Projections of the male pelvic anatomy relative to a set of original 3D CT images 201 are shown in FIG. 2B. Selected organs at risk and target organs were contoured in the 3D CT image 201 and their voxels were assigned a code value depending on the type of anatomy. Projection images 250 at selected angles (0 degrees, 45 degrees, and 90 degrees) about the central axis of the 3D CT image 201 can be obtained using the forward projection capability of a reconstruction process (e.g., a cone beam CT reconstruction program). Projection images can also be computed either by directly re-creating the projection view geometry by ray tracing or by Fourier reconstruction such as is used in computed tomography.

In an example, the projection image can be computed by tracing the path of light as pixels in an image plane and simulating the effects of its encounters with virtual objects. In some implementations, the projection image is generated by tracing a path from an imaginary eye (a beam's eye view, or an MLC view) through each pixel in a virtual screen and calculating the color of the object visible through it. Other tomographic reconstruction techniques can be utilized to generate the projection images from the views of the anatomy depicted in the 3D CT images 201.

For example, the set of (or collection of) 3D CT images 201 can be used to generate one or more views of the anatomy (e.g., the bladder, prostate, seminal vesicles, rectum, first and second targets) depicted in the 3D CT images 201. The views can be from the perspective of the radiotherapy beam (e.g., as provided by the gantry of the radiotherapy device) and, for simplicity with reference to FIG. 2B, the views are measured in degrees relative to the y-axis of the 3D CT images 201 and based on a distance between the anatomy depicted in the image and the MLC. Specifically, a first view 210 represents a projection of the 3D CT images 201 when viewed or seen from the gantry when the gantry is 0 degrees relative to the y-axis and is at a given distance from the anatomy depicted in the 3D CT image 201, a second view 220 represents a projection of the 3D CT images 201 when viewed or seen by the gantry when the gantry is 45 degrees relative to the y-axis and is at a given distance from the anatomy depicted in the 3D CT image 201, and a third view 230 represents a projection of the 3D CT images 201 when viewed or seen by the gantry when the gantry is 90 degrees relative to the y-axis. Any other views can be provided, such as a different view at each of 360 degrees around the anatomy depicted in the 3D CT images 201.

Referring back to FIG. 1, in yet another example, the software programs 144 may generate graphical image representations of control point data (variously referred to as control point representations, control point images, or “control points”) at various radiotherapy beam and gantry angles, using the machine learning techniques discussed herein. In particular, the software programs 144 may optimize information from these control point representations in machine learning-assisted aspects of arc sequencing and direct aperture optimization. Such control point data, when refined and optimized as appropriate, will control a radiotherapy device to produce a radiotherapy beam. The control points may represent the beam intensity, gantry angle relative to the patient position, and the leaf positions of the MLC, among other machine parameters, to deliver a radiotherapy dose.

In yet another example, the software programs 144 store a treatment planning software that includes a trained machine learning model, such as a trained generative model from a generative adversarial network (GAN), or a conditional generative adversarial network (cGAN) to generate or estimate a control point image at a given radiotherapy beam angle, based on input to the model of a projection image of the anatomy representing the view of the anatomy from the given angle, and the treatment constraints (e.g., target doses and organs at risk) in such anatomy. The software programs 144 may further store a function to optimize or accept further optimization of the control point data, and to convert or translate the control point data into other formats or parameters for a given type of radiotherapy machine (e.g., to output a beam from a MLC to achieve a particular dosage using the MLC leaf positions). As a result, the treatment planning software may perform a number of computations to adapt the beam shape and intensity for each radiotherapy beam and gantry angle to the radiotherapy treatment constraints, and to compute the control points for a given radiotherapy device to achieve that beam shape and intensity in the subject patient.

In addition to the memory device 116 storing the software programs 144, it is contemplated that software programs 144 may be stored on a removable computer medium, such as a hard drive, a computer disk, a CD-ROM, a DVD, a HD, a Blu-Ray DVD, USB flash drive, a SD card, a memory stick, or any other suitable medium; and the software programs 144 when downloaded to image processing device 112 may be executed by image processor 114.

The processor 114 may be communicatively coupled to the memory device 116, and the processor 114 may be configured to execute computer-executable instructions stored thereon. The processor 114 may send or receive medical images 146 to memory device 116. For example, the processor 114 may receive medical images 146 from the image acquisition device 132 via the communication interface 118 and network 120 to be stored in memory device 116. The processor 114 may also send medical images 146 stored in memory device 116 via the communication interface 118 to the network 120 to be either stored in database 124 or the hospital database 126.

Further, the processor 114 may utilize software programs 144 (e.g., a treatment planning software) along with the medical images 146 and patient data 145 to create the radiation therapy treatment plan 142. Medical images 146 may include information such as imaging data associated with a patient anatomical region, organ, or volume of interest segmentation data. Patient data 145 may include information such as (1) functional organ modeling data (e.g., serial versus parallel organs, appropriate dose response models, etc.); (2) radiation dosage data (e.g., DVH information; or (3) other clinical information about the patient and course of treatment (e.g., other surgeries, chemotherapy, previous radiotherapy, etc.).

In addition, the processor 114 may utilize software programs to generate intermediate data such as updated parameters to be used, for example, by a machine learning model, such as a neural network model; or generate intermediate 2D or 3D images, which may then subsequently be stored in memory device 116. The processor 114 may subsequently then transmit the executable radiation therapy treatment plan 142 via the communication interface 118 to the network 120 to the radiation therapy device 130, where the radiation therapy plan will be used to treat a patient with radiation. In addition, the processor 114 may execute software programs 144 to implement functions such as image conversion, image segmentation, deep learning, neural networks, and artificial intelligence. For instance, the processor 114 may execute software programs 144 that train or contour a medical image; such software programs 144 when executed may train a boundary detector or utilize a shape dictionary.

The processor 114 may be a processing device, include one or more general-purpose processing devices such as a microprocessor, a central processing unit (CPU), a graphics processing unit (GPU), an accelerated processing unit (APU), or the like. More particularly, the processor 114 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processor 114 may also be implemented by one or more special-purpose processing devices such as an application-specific integrated circuit (ASIC), a field programmable gate array (FPGA), a digital signal processor (DSP), a System on a Chip (SoC), or the like. As would be appreciated by those skilled in the art, in some examples, the processor 114 may be a special-purpose processor, rather than a general-purpose processor. The processor 114 may include one or more known processing devices, such as a microprocessor from the Pentium™, Core™, Xeon™, or Itanium® family manufactured by Intel™, the Turion™, Athlon™, Sempron™, Opteron™ FX™, Phenom™ family manufactured by AMD™, or any of various processors manufactured by Sun Microsystems. The processor 114 may also include graphical processing units such as a GPU from the GeForce®, Quadro®, Tesla® family manufactured by Nvidia™, GMA, Iris™ family manufactured by Intel™ or the Radeon™ family manufactured by AMD™. The processor 114 may also include accelerated processing units such as the Xeon Phi™ family manufactured by Intel™. The disclosed examples are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor (for example, a multi-core design or a plurality of processors each having a multi-core design). The processor 114 can execute sequences of computer program instructions, stored in memory device 116, to perform various operations, processes, methods that will be explained in greater detail below.

The memory device 116 can store medical images 146. In some examples, the medical images 146 may include one or more MRI images (e.g., 2D MRI, 3D MRI, 2D streaming MRI, four-dimensional (4D) MRI, 4D volumetric MRI, 4D cine MRI, projection images, fluence map representation images, pairing information between projection (anatomy or treatment) images and fluence map representation images, aperture representation (control point) images or data representations, pairing information between projection (anatomy or treatment) images and aperture (control point) images or representations, functional MRI images (e.g., fMRI, DCE-MRI, diffusion MRI), CT images (e.g., 2D CT, cone beam CT, 3D CT, 4D CT), ultrasound images (e.g., 2D ultrasound, 3D ultrasound, 4D ultrasound), one or more projection images representing views of an anatomy depicted in the MRI, synthetic CT (pseudo-CT), and/or CT images at different angles of a gantry relative to a patient axis, PET images, X-ray images, fluoroscopic images, radiotherapy portal images, SPECT images, computer generated synthetic images (e.g., pseudo-CT images), aperture images, graphical aperture image representations of MLC leaf positions at different gantry angles, and the like. Further, the medical images 146 may also include medical image data, for instance, training images, and training images, contoured images, and dose images. In an example, the medical images 146 may be received from the image acquisition device 132. Accordingly, image acquisition device 132 may include an MRI imaging device, a CT imaging device, a PET imaging device, an ultrasound imaging device, a fluoroscopic device, a SPECT imaging device, an integrated linac and MRI imaging device, or other medical imaging devices for obtaining the medical images of the patient. The medical images 146 may be received and stored in any type of data or any type of format that the image processing device 112 may use to perform operations consistent with the disclosed examples.

The memory device 116 may be a non-transitory computer-readable medium, such as a read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a CD-ROM, a DVD or other optical storage, a cassette tape, other magnetic storage device, or any other non-transitory medium that may be used to store information including image, data, or computer executable instructions (e.g., stored in any format) capable of being accessed by the processor 114, or any other type of computer device. The computer program instructions can be accessed by the processor 114, read from the ROM, or any other suitable memory location, and loaded into the RAM for execution by the processor 114. For example, the memory device 116 may store one or more software applications. Software applications stored in the memory device 116 may include, for example, an operating system 143 for common computer systems as well as for software-controlled devices. Further, the memory device 116 may store an entire software application, or only a part of a software application, that are executable by the processor 114. For example, the memory device 116 may store one or more radiation therapy treatment plans 142.

The image processing device 112 can communicate with the network 120 via the communication interface 118, which can be communicatively coupled to the processor 114 and the memory device 116. The communication interface 118 may provide communication connections between the image processing device 112 and radiotherapy system 100 components (e.g., permitting the exchange of data with external devices). For instance, the communication interface 118 may in some examples have appropriate interfacing circuitry to connect to the user interface 136, which may be a hardware keyboard, a keypad, or a touch screen through which a user may input information into radiotherapy system 100.

Communication interface 118 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a Wi-Fi adaptor), a telecommunication adaptor (e.g., 3G, 4G/LTE and the like), and the like. Communication interface 118 may include one or more digital and/or analog communication devices that permit image processing device 112 to communicate with other machines and devices, such as remotely located components, via the network 120.

The network 120 may provide the functionality of a local area network (LAN), a wireless network, a cloud computing environment (e.g., software as a service, platform as a service, infrastructure as a service, etc.), a client-server, a wide area network (WAN), and the like. For example, network 120 may be a LAN or a WAN that may include other systems S1 (138), S2 (140), and S3 (141). Systems S1, S2, and S3 may be identical to image processing device 112 or may be different systems. In some examples, one or more of the systems in network 120 may form a distributed computing/simulation environment that collaboratively performs the examples described herein. In some examples, one or more systems S1, S2, and S3 may include a CT scanner that obtains CT images (e.g., medical images 146). In addition, network 120 may be connected to Internet 122 to communicate with servers and clients that reside remotely on the Internet.

Therefore, network 120 can allow data transmission between the image processing device 112 and a number of various other systems and devices, such as the OIS 128, the radiation therapy device 130, and the image acquisition device 132. Further, data generated by the OIS 128 and/or the image acquisition device 132 may be stored in the memory device 116, the database 124, and/or the hospital database 126. The data may be transmitted/received via network 120, through communication interface 118 in order to be accessed by the processor 114, as required.

The image processing device 112 may communicate with database 124 through network 120 to send/receive a plurality of various types of data stored on database 124. For example, database 124 may include machine data (control points) that includes information associated with a radiation therapy device 130, image acquisition device 132, or other machines relevant to radiotherapy. Machine data information may include control points, such as radiation beam size, arc placement, beam on and off time duration, machine parameters, segments, MLC configuration, gantry speed, MRI pulse sequence, and the like. Database 124 may be a storage device and may be equipped with appropriate database administration software programs. One skilled in the art would appreciate that database 124 may include a plurality of devices located either in a central or a distributed manner.

In some examples, database 124 may include a processor-readable storage medium (not shown). While the processor-readable storage medium in an example may be a single medium, the term “processor-readable storage medium” should be taken to include a single medium or multiple media (e.g., a centralized or distributed database, and/or associated caches and servers) that store the one or more sets of computer-executable instructions or data. The term “processor-readable storage medium” shall also be taken to include any medium that is capable of storing or encoding a set of instructions for execution by a processor and that cause the processor to perform any one or more of the methodologies of the present disclosure. The term “processor-readable storage medium” shall accordingly be taken to include, but not be limited to, solid-state memories, optical and magnetic media. For example, the processor-readable storage medium can be one or more volatile, non-transitory, or non-volatile tangible computer-readable media.

Image processor 114 may communicate with database 124 to read images into memory device 116 or store images from memory device 116 to database 124. For example, the database 124 may be configured to store a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DICOM) data, projection images, graphical aperture images, etc.) that the database 124 received from image acquisition device 132. Database 124 may store data to be used by the image processor 114 when executing software program 144, or when creating radiation therapy treatment plans 142. Database 124 may store the data produced by the trained machine leaning mode, such as a neural network including the network parameters constituting the model learned by the network and the resulting predicted data. The image processing device 112 may receive the imaging data, such as a medical image 146 (e.g., 2D MRI slice images, CT images, 2D Fluoroscopy images, X-ray images, 3D MRI images, 4D MRI images, projection images, graphical aperture images, etc.) either from the database 124, the radiation therapy device 130 (e.g., an MRI-linac), and/or the image acquisition device 132 to generate a radiation therapy treatment plan 142.

In an example, the radiotherapy system 100 can include an image acquisition device 132 that can acquire medical images (e.g., MRI images, 3D MRI, 2D streaming MRI, 4D volumetric MRI, CT images, cone-Beam CT, PET images, functional MRI images (e.g., fMRI, DCE-MRI and diffusion MRI), X-ray images, fluoroscopic image, ultrasound images, radiotherapy portal images, SPECT images, and the like) of the patient. Image acquisition device 132 may, for example, be an MRI imaging device, a CT imaging device, a PET imaging device, an ultrasound device, a fluoroscopic device, a SPECT imaging device, or any other suitable medical imaging device for obtaining one or more medical images of the patient. Images acquired by the image acquisition device 132 can be stored within database 124 as either imaging data and/or test data. By way of example, the images acquired by the image acquisition device 132 can also be stored by the image processing device 112, as medical images 146 in memory device 116.

In an example, the image acquisition device 132 may be integrated with the radiation therapy device 130 as a single apparatus (e.g., an MRI-linac). Such an MRI-linac can be used, for example, to determine a location of a target organ or a target tumor in the patient, so as to direct radiation therapy accurately according to the radiation therapy treatment plan 142 to a predetermined target.

The image acquisition device 132 can be configured to acquire one or more images of the patient's anatomy for a region of interest (e.g., a target organ, a target tumor, or both). Each image, typically a 2D image or slice, can include one or more parameters (e.g., a 2D slice thickness, an orientation, and a location, etc.). In an example, the image acquisition device 132 can acquire a 2D slice in any orientation. For example, an orientation of the 2D slice can include a sagittal orientation, a coronal orientation, or an axial orientation. The processor 114 can adjust one or more parameters, such as the thickness and/or orientation of the 2D slice, to include the target organ and/or target tumor. In an example, 2D slices can be determined from information such as a 3D MRI volume. Such 2D slices can be acquired by the image acquisition device 132 in “real-time” while a patient is undergoing radiation therapy treatment, for example, when using the radiation therapy device 130, with “real-time” meaning acquiring the data in at least milliseconds or less.

The image processing device 112 may generate and store radiation therapy treatment plans 142 for one or more patients. The radiation therapy treatment plans 142 may provide information about a particular radiation dose to be applied to each patient. The radiation therapy treatment plans 142 may also include other radiotherapy information, such as control points including beam angles, gantry angles, beam intensity, dose-histogram-volume information, the number of radiation beams to be used during therapy, the dose per beam, and the like.

The image processor 114 may generate the radiation therapy treatment plan 142 by using software programs 144 such as treatment planning software (such as Monaco®, manufactured by Elekta AB of Stockholm, Sweden). In order to generate the radiation therapy treatment plans 142, the image processor 114 may communicate with the image acquisition device 132 (e.g., a CT device, an MRI device, a PET device, an X-ray device, an ultrasound device, etc.) to access images of the patient and to delineate a target, such as a tumor. In some examples, the delineation of one or more OARs, such as healthy tissue surrounding the tumor or in close proximity to the tumor may be required. Therefore, segmentation of the OAR may be performed when the OAR is close to the target tumor. In addition, if the target tumor is close to the OAR (e.g., prostate in near proximity to the bladder and rectum), then by segmenting the OAR from the tumor, the radiotherapy system 100 may study the dose distribution not only in the target but also in the OAR.

In order to delineate a target organ or a target tumor from the OAR, medical images, such as MRI images, CT images, PET images, fMRI images, X-ray images, ultrasound images, radiotherapy portal images, SPECT images, and the like, of the patient undergoing radiotherapy may be obtained non-invasively by the image acquisition device 132 to reveal the internal structure of a body part. Based on the information from the medical images, a 3D structure of the relevant anatomical portion may be obtained. In addition, during a treatment planning process, many parameters may be taken into consideration to achieve a balance between efficient treatment of the target tumor (e.g., such that the target tumor receives enough radiation dose for an effective therapy) and low irradiation of the OAR(s) (e.g., the OAR(s) receives as low a radiation dose as possible). Other parameters that may be considered include the location of the target organ and the target tumor, the location of the OAR, and the movement of the target in relation to the OAR. For example, the 3D structure may be obtained by contouring the target or contouring the OAR within each 2D layer or slice of an MRI or CT image and combining the contour of each 2D layer or slice. The contour may be generated manually (e.g., by a physician, dosimetrist, or health care worker using a program such as MONACO™ manufactured by Elekta AB of Stockholm, Sweden) or automatically (e.g., using a program such as the Atlas-based auto-segmentation software, ABAS™, and a successor auto-segmentation software product ADMIRE™, manufactured by Elekta AB of Stockholm, Sweden). In certain examples, the 3D structure of a target tumor or an OAR may be generated automatically by the treatment planning software.

After the target tumor and the OAR(s) have been located and delineated, a dosimetrist, physician, or healthcare worker may determine a dose of radiation to be applied to the target tumor, as well as any maximum amounts of dose that may be received by the OAR proximate to the tumor (e.g., left and right parotid, optic nerves, eyes, lens, inner ears, spinal cord, brain stem, and the like). After the radiation dose is determined for each anatomical structure (e.g., target tumor, OAR), a process known as inverse planning may be performed to determine one or more treatment plan parameters that would achieve the desired radiation dose distribution. Examples of treatment plan parameters include volume delineation parameters (e.g., which define target volumes, contour sensitive structures, etc.), margins around the target tumor and OARs, beam angle selection, collimator settings, and beam-on times. During the inverse-planning process, the physician may define dose constraint parameters that set bounds on how much radiation an OAR may receive (e.g., defining full dose to the tumor target and zero dose to any OAR; defining 95% of dose to the target tumor; defining that the spinal cord, brain stem, and optic structures receive ≤45 Gy, ≤55 Gy and <54 Gy, respectively). The result of inverse planning may constitute a radiation therapy treatment plan 142 that may be stored in memory device 116 or database 124. Some of these treatment parameters may be correlated. For example, tuning one parameter (e.g., weights for different objectives, such as increasing the dose to the target tumor) in an attempt to change the treatment plan may affect at least one other parameter, which in turn may result in the development of a different treatment plan. Thus, the image processing device 112 can generate a tailored radiation therapy treatment plan 142 having these parameters in order for the radiation therapy device 130 to provide radiotherapy treatment to the patient.

In addition, the radiotherapy system 100 may include a display device 134 and a user interface 136. The display device 134 may include one or more display screens that display medical images, interface information, treatment planning parameters (e.g., projection images, graphical aperture images, contours, dosages, beam angles, etc.) treatment plans, a target, localizing a target and/or tracking a target, or any related information to the user. The user interface 136 may be a keyboard, a keypad, a touch screen or any type of device with which a user may input information to radiotherapy system 100. Alternatively, the display device 134 and the user interface 136 may be integrated into a device such as a tablet computer (e.g., Apple iPad®, Lenovo Thinkpad®, Samsung Galaxy®, etc.).

Furthermore, any and all components of the radiotherapy system 100 may be implemented as a virtual machine (e.g., VMWare, Hyper-V, and the like). For instance, a virtual machine can be software that functions as hardware. Therefore, a virtual machine can include at least one or more virtual processors, one or more virtual memories, and one or more virtual communication interfaces that together function as hardware. For example, the image processing device 112, the OIS 128, the image acquisition device 132 could be implemented as a virtual machine. Given the processing power, memory, and computational capability available, the entire radiotherapy system 100 could be implemented as a virtual machine.

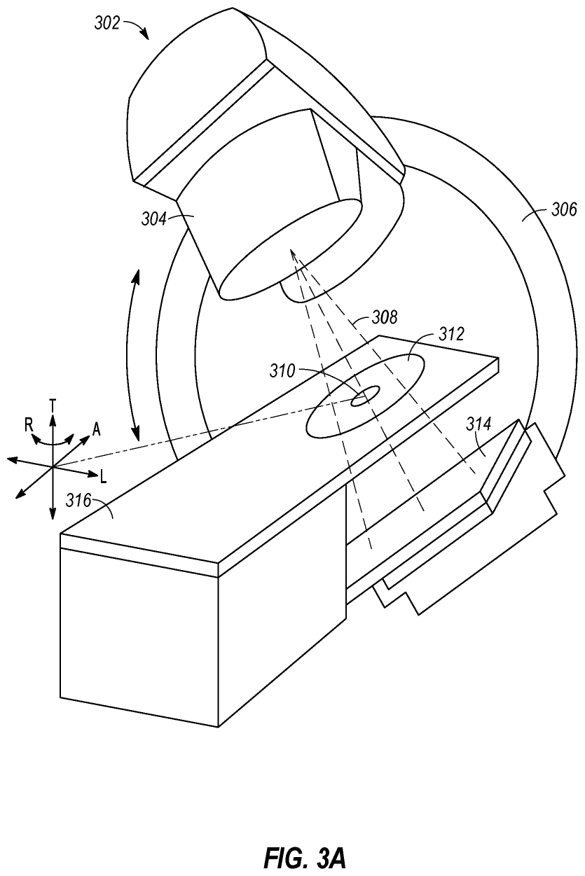

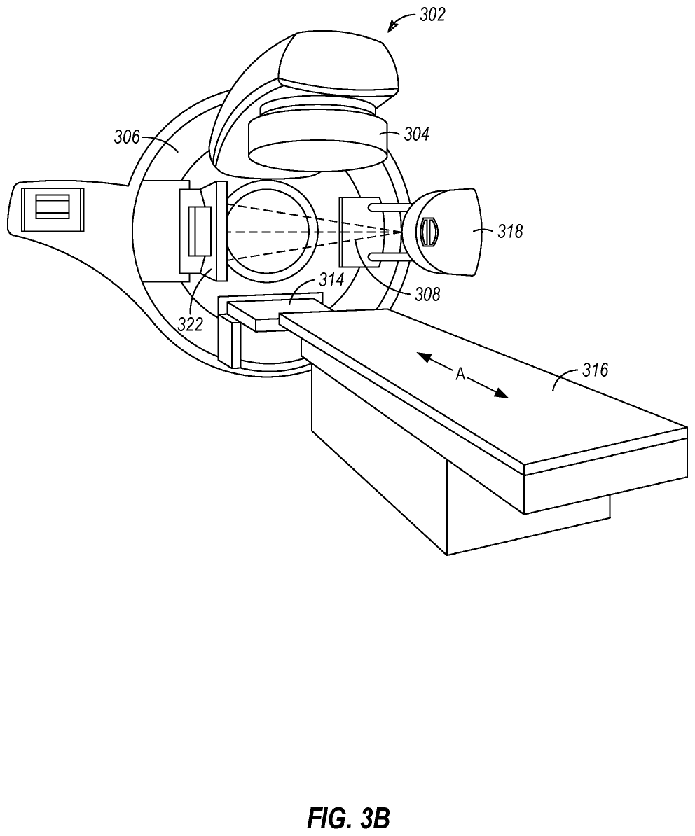

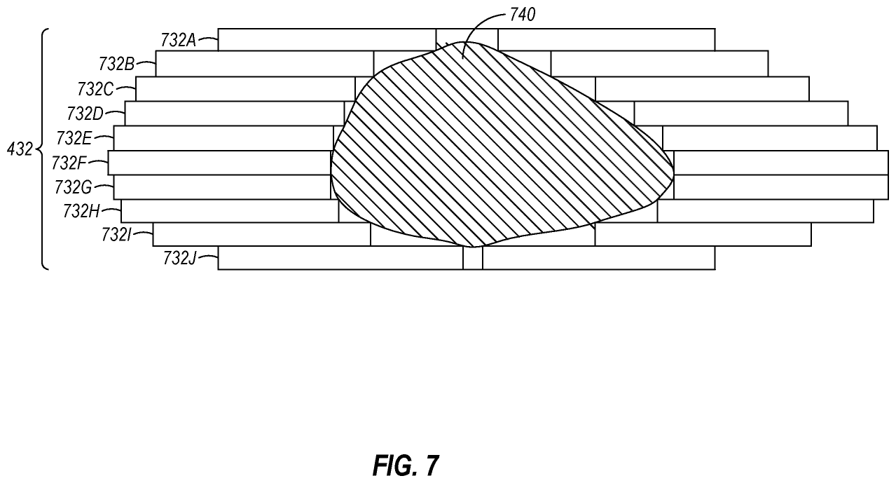

FIG. 3A illustrates a radiation therapy device 302 that may include a radiation source, such as an X-ray source or a linear accelerator, a couch 316, an imaging detector 314, and a radiation therapy output 304. The radiation therapy device 302 may be configured to emit a radiation beam 308 to provide therapy to a patient. The radiation therapy output 304 can include one or more attenuators or collimators, such as an MLC as described in the illustrative example of FIG. 7, below.

Referring back to FIG. 3A, a patient can be positioned in a region 312 and supported by the treatment couch 316 to receive a radiation therapy dose, according to a radiation therapy treatment plan. The radiation therapy output 304 can be mounted or attached to a gantry 306 or other mechanical support. One or more chassis motors (not shown) may rotate the gantry 306 and the radiation therapy output 304 around couch 316 when the couch 316 is inserted into the treatment area. In an example, gantry 306 may be continuously rotatable around couch 316 when the couch 316 is inserted into the treatment area. In another example, gantry 306 may rotate to a predetermined position when the couch 316 is inserted into the treatment area. For example, the gantry 306 can be configured to rotate the therapy output 304 around an axis (“A”). Both the couch 316 and the radiation therapy output 304 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 316 movements or rotations in order to properly position the patient in or out of the radiation beam 308 according to a radiation therapy treatment plan. Both the couch 316 and the gantry 306 are independently moveable from one another in multiple degrees of freedom, which allows the patient to be positioned such that the radiation beam 308 can target the tumor precisely. The MLC may be integrated and included within gantry 306 to deliver the radiation beam 308 of a certain shape.

The coordinate system (including axes A, T, and L) shown in FIG. 3A can have an origin located at an isocenter 310. The isocenter can be defined as a location where the central axis of the radiation beam 308 intersects the origin of a coordinate axis, such as to deliver a prescribed radiation dose to a location on or within a patient. Alternatively, the isocenter 310 can be defined as a location where the central axis of the radiation beam 308 intersects the patient for various rotational positions of the radiation therapy output 304 as positioned by the gantry 306 around the axis A. As discussed herein, the gantry angle corresponds to the position of gantry 306 relative to axis A, although any other axis or combination of axes can be referenced and used to determine the gantry angle.

Gantry 306 may also have an attached imaging detector 314. The imaging detector 314 is preferably located opposite to the radiation source, and in an example, the imaging detector 314 can be located within a field of the radiation beam 308.

The imaging detector 314 can be mounted on the gantry 306 (preferably opposite the radiation therapy output 304), such as to maintain alignment with the therapy beam 308. The imaging detector 314 rotates about the rotational axis as the gantry 306 rotates. In an example, the imaging detector 314 can be a flat panel detector (e.g., a direct detector or a scintillator detector). In this manner, the imaging detector 314 can be used to monitor the radiation beam 308 or the imaging detector 314 can be used for imaging the patient's anatomy, such as portal imaging. The control circuitry of the radiation therapy device 302 may be integrated within the radiotherapy system 100 or remote from it.

In an illustrative example, one or more of the couch 316, the therapy output 304, or the gantry 306 can be automatically positioned, and the therapy output 304 can establish the radiation beam 308 according to a specified dose for a particular therapy delivery instance. A sequence of therapy deliveries can be specified according to a radiation therapy treatment plan, such as using one or more different orientations or locations of the gantry 306, couch 316, or therapy output 304. The therapy deliveries can occur sequentially, but can intersect in a desired therapy locus on or within the patient, such as at the isocenter 310. A prescribed cumulative dose of radiation therapy can thereby be delivered to the therapy locus while damage to tissue near the therapy locus can be reduced or avoided.

FIG. 3B illustrates a radiation therapy device 302 that may include a combined linac and an imaging system, such as a CT imaging system. The radiation therapy device 302 can include an MLC (not shown). The CT imaging system can include an imaging X-ray source 318, such as providing X-ray energy in a kiloelectron-Volt (keV) energy range. The imaging X-ray source 318 can provide a fan-shaped and/or a conical radiation beam 308 directed to an imaging detector 322, such as a flat panel detector. The radiation therapy device 302 can be similar to the system described in relation to FIG. 3A, such as including a radiation therapy output 304, a gantry 306, a couch 316, and another imaging detector 314 (such as a flat panel detector). The X-ray source 318 can provide a comparatively-lower-energy X-ray diagnostic beam, for imaging.

In the illustrative example of FIG. 3B, the radiation therapy output 304 and the X-ray source 318 can be mounted on the same rotating gantry 306, rotationally separated from each other by 90 degrees. In another example, two or more X-ray sources can be mounted along the circumference of the gantry 306, such as each having its own detector arrangement to provide multiple angles of diagnostic imaging concurrently. Similarly, multiple radiation therapy outputs 304 can be provided.

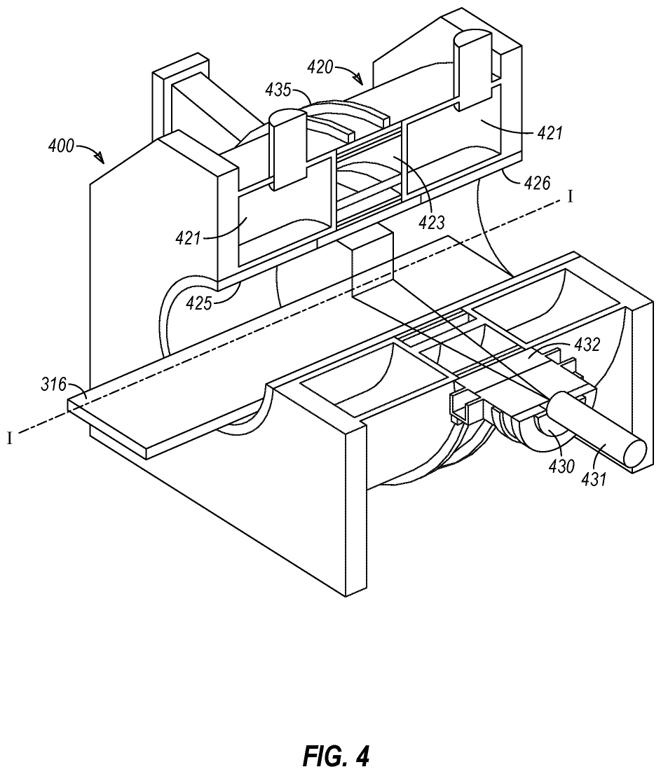

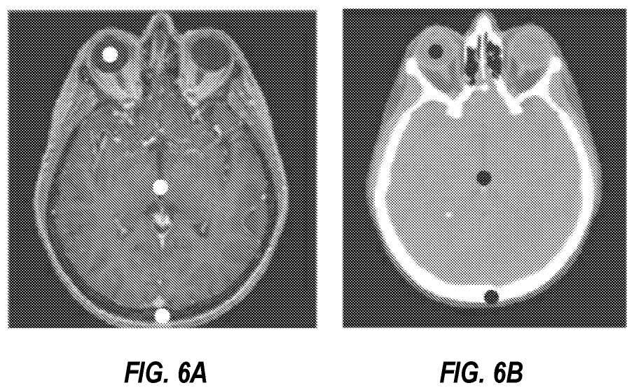

FIG. 4 depicts a radiation therapy system 400 that can include combining a radiation therapy device 302 and an imaging system, such as a magnetic resonance (MR) imaging system (e.g., known in the art as an MR-linac) consistent with the disclosed examples. As shown, system 400 may include a couch 316, an image acquisition device 420, and a radiation delivery device 430. System 400 delivers radiation therapy to a patient in accordance with a radiotherapy treatment plan. In some examples, image acquisition device 420 may correspond to image acquisition device 132 in FIG. 1 that may acquire origin images of a first modality (e.g., MRI image shown in FIG. 6A) or destination images of a second modality (e.g., CT image shown in FIG. 6B).

Couch 316 may support a patient (not shown) during a treatment session. In some implementations, couch 316 may move along a horizontal translation axis (labelled “I”), such that couch 316 can move the patient resting on couch 316 into and/or out of system 400. Couch 316 may also rotate around a central vertical axis of rotation, transverse to the translation axis. To allow such movement or rotation, couch 316 may have motors (not shown) enabling the couch 316 to move in various directions and to rotate along various axes. A controller (not shown) may control these movements or rotations in order to properly position the patient according to a treatment plan.

In some examples, image acquisition device 420 may include an MRI machine used to acquire 2D or 3D MRI images of the patient before, during, and/or after a treatment session. Image acquisition device 420 may include a magnet 421 for generating a primary magnetic field for magnetic resonance imaging. The magnetic field lines generated by operation of magnet 421 may run substantially parallel to the central translation axis I. Magnet 421 may include one or more coils with an axis that runs parallel to the translation axis I. In some examples, the one or more coils in magnet 421 may be spaced such that a central window 423 of magnet 421 is free of coils. In other examples, the coils in magnet 421 may be thin enough or of a reduced density such that they are substantially transparent to radiation of the wavelength generated by radiotherapy device 430. Image acquisition device 420 may also include one or more shielding coils, which may generate a magnetic field outside magnet 421 of approximately equal magnitude and opposite polarity in order to cancel or reduce any magnetic field outside of magnet 421. As described below, radiation source 431 of radiation delivery device 430 may be positioned in the region where the magnetic field is cancelled, at least to a first order, or reduced.

Image acquisition device 420 may also include two gradient coils 425 and 426, which may generate a gradient magnetic field that is superposed on the primary magnetic field. Coils 425 and 426 may generate a gradient in the resultant magnetic field that allows spatial encoding of the protons so that their position can be determined. Gradient coils 425 and 426 may be positioned around a common central axis with the magnet 421 and may be displaced along that central axis. The displacement may create a gap, or window, between coils 425 and 426. In examples where magnet 421 can also include a central window 423 between coils, the two windows may be aligned with each other.

In some examples, image acquisition device 420 may be an imaging device other than an MRI, such as an X-ray, a CT, a CBCT, a spiral CT, a PET, a SPECT, an optical tomography, a fluorescence imaging, ultrasound imaging, radiotherapy portal imaging device, or the like. As would be recognized by one of ordinary skill in the art, the above description of image acquisition device 420 concerns certain examples and is not intended to be limiting.

Radiation delivery device 430 may include the radiation source 431, such as an X-ray source or a linac, and an MLC 432 (shown below in more detail in FIG. 7). Radiation delivery device 430 may be mounted on a chassis 435. One or more chassis motors (not shown) may rotate the chassis 435 around the couch 316 when the couch 316 is inserted into the treatment area. In an example, the chassis 435 may be continuously rotatable around the couch 316, when the couch 316 is inserted into the treatment area. Chassis 435 may also have an attached radiation detector (not shown), preferably located opposite to radiation source 431 and with the rotational axis of the chassis 435 positioned between the radiation source 431 and the detector. Further, the device 430 may include control circuitry (not shown) used to control, for example, one or more of the couch 316, image acquisition device 420, and radiotherapy device 430. The control circuitry of the radiation delivery device 430 may be integrated within the system 400 or remote from it.

During a radiotherapy treatment session, a patient may be positioned on couch 316. System 400 may then move couch 316 into the treatment area defined by the magnet 421, coils 425, 426, and chassis 435. Control circuitry may then control radiation source 431, MLC 432, and the chassis motor(s) to deliver radiation to the patient through the window between coils 425 and 426 according to a radiotherapy treatment plan.

FIG. 3A, FIG. 3B, and FIG. 4 generally illustrate examples of a radiation therapy device configured to provide radiotherapy treatment to a patient, including a configuration where a radiation therapy output can be rotated around a central axis (e.g., an axis “A”). Other radiation therapy output configurations can be used. For example, a radiation therapy output can be mounted to a robotic arm or manipulator having multiple degrees of freedom. In yet another example, the therapy output can be fixed, such as located in a region laterally separated from the patient, and a platform supporting the patient can be used to align a radiation therapy isocenter with a specified target locus within the patient.

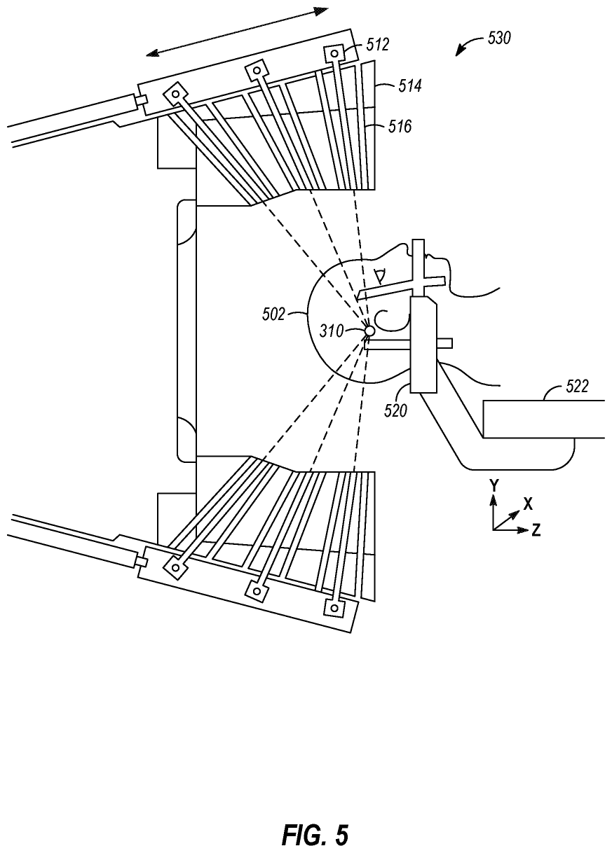

FIG. 5 illustrates an example of another type of radiotherapy device 530 (e.g., a Leksell Gamma Knife). As shown in FIG. 5, in a radiotherapy treatment session, a patient 502 may wear a coordinate frame 520 to keep stable the patient's body part (e.g., the head) undergoing surgery or radiotherapy. Coordinate frame 520 and a patient positioning system 522 may establish a spatial coordinate system, which may be used while imaging a patient or during radiation surgery. Radiotherapy device 530 may include a protective housing 514 to enclose a plurality of radiation sources 512. Radiation sources 512 may generate a plurality of radiation beams (e.g., beamlets) through beam channels 516. The plurality of radiation beams may be configured to focus on an isocenter 310 from different directions. While each individual radiation beam may have a relatively low intensity, isocenter 310 may receive a relatively high level of radiation when multiple doses from different radiation beams accumulate at isocenter 310. In certain examples, isocenter 310 may correspond to a target under surgery or treatment, such as a tumor.

As discussed above, radiation therapy devices described by FIG. 3A, FIG. 3B, and FIG. 4 include an MLC for shaping, directing, or modulating an intensity of a radiation therapy beam to the specified target locus within the patient. FIG. 7 illustrates an MLC 432 that includes leaves 732A through 732J that can be automatically positioned to define an aperture approximating a tumor 740 cross-section or projection. The leaves 732A through 732J permit modulation of the radiation therapy beam. The leaves 732A through 732J can be made of a material specified to attenuate or block the radiation beam in regions other than the aperture, in accordance with the radiation treatment plan. For example, the leaves 732A through 732J can include metallic plates, such as comprising tungsten, with a long axis of the plates oriented parallel to a beam direction and having ends oriented orthogonally to the beam direction (as shown in the plane of the illustration of FIG. 2A). A “state” of the MLC 432 can be adjusted adaptively during a course of radiation therapy treatment, such as to establish a therapy beam that better approximates a shape or location of the tumor 740 or other target locus. This is in comparison to using a static collimator configuration or as compared to using an MLC configuration determined exclusively using an “offline” therapy planning technique. A radiation therapy technique using the MLC 432 to produce a specified radiation dose distribution to a tumor or to specific areas within a tumor can be referred to as IMRT. The resulting beam shape that is output using the MLC 432 is represented as a graphical aperture image. Namely, a given graphical aperture image is generated to represent how a beam looks (beam shape) and its intensity after being passed through and output by MLC 432.

IMRT planning proceeds through two stages: 1) the creation of a fluence map optimally depositing energy on the target while sparing surrounding OARs, and 2) the translation of the fluences for each beam into a sequence of multileaf collimator (MLC) apertures that shape the beam boundary and modulate its intensity profile. This is the basic procedure for step-and-shoot IMRT. It is in Stage 1 that the planning must resolve the conflicting constraints for prescribed target dose and organ sparing, as fluence map optimization considers the treatment planning features and constraint conflicts. Stage 2 transforms the optimal fluence map into sets of machine parameters, called control points, that specify to the treatment linear accelerator (linac) equipped with an MLC, how the target is to be irradiated. The reduction of the treatment goals and optimal fluence to efficiently deliverable MLC apertures (segments) is called segmentation. The control points define how each beam (IMRT) or arc sector (VMAT) is to be delivered. Each control point consists of the given beam's gantry angle, the set of MLC leaf-edge positions, and the total monitor units (MUs, beam fluence) delivered in all previous control points.

The MLC leaf edges collectively define the beam aperture, the beam's-eye view of the target. The aperture is discretized into a rectangular grid perpendicular to the beam direction, defined by the spacing and travel settings of the MLC leaves. The portion of a treatment beam admitted through an aperture element is called a beamlet. An aperture beamlet pixel, or bixel, transmits zero X-ray fluence when blocked by a jaw or a leaf, and transmits some fluence when partly or fully unblocked. The amount of fluence depends on the dose rate or the beam-on time during which constant fluence is transmitted through this bixel. Multiple apertures with different bixel patterns may be created at the same angle to provide a non-uniform fluence profile, called fluence modulation.

IMRT techniques involve irradiating a subject patient at a small number of fixed gantry angles; whereas VMAT techniques typically involve irradiating a subject patient from 100 or more gantry angles. Specifically, with VMAT radiotherapy devices, the patient is irradiated continuously by a linac revolving around the patient with a beam continuously shaped by MLC producing apertures to achieve a modulated coverage of the target, from each angle, by a prescribed radiation dose. VMAT has become popular because it accurately irradiates targets while minimizing dose to neighboring OARs, and VMAT treatments generally take less time than those of IMRT.

As noted above, in IMRT, the optimal set of control point quantities is obtained in a two-step procedure: 1) find the optimal map of X-ray fluence (intensity) over the target by varying the directions and shapes of the beams, and 2) find the set of MLC-deliverable apertures (sequencing) that deliver a dose distribution over the target that most closely approximates the optimal fluence map. Typical IMRT treatments are delivered in 5-9 discrete beams. For VMAT, the optimal set of control point quantities is obtained by a variant of the following three-step procedure: 1) optimize the fluence map for a fixed set of static beams spaced q-degrees apart; 2) sequence each fluence map into apertures spaced equidistantly over the q-degree arc sector, and 3) refine the apertures by optimizing over the leaf positions and aperture intensities. The third step is known as direct aperture optimization (DAO).

VMAT has substantially shorter delivery times than IMRT, since the gantry and the MLC leaves are in continuous motion during treatment. For IMRT, the gantry drives to each beam's gantry angle in turn, stops, and delivers the aperture-modulated beam while the gantry remains stationary. VMAT delivery times may be a factor of 12 or less of those of IMRT.

Creating plans personalized for every patient using either IMRT or VMAT is difficult. Treatment planning systems generally model the physics of a radiation dose, but they provide little assistance to the planner to indicate how to vary treatment parameters to achieve high quality plans. Changing plan variables often produces nonintuitive results, and the treatment planning system is unable to tell the planner whether a little or a lot of effort will be needed to advance the current plan-in-progress to a clinically usable plan. Automated multicriteria optimization reduces planning uncertainty by automated, exhaustive numerical optimizations satisfying a hierarchy of target-OAR constraints, but this method is time consuming and often does not produce a deliverable plan.

In VMAT, the patient is treated by radiation passing through the control point apertures with the intensities specified by the control point meterset weights or monitor units, at each of a series of LINAC gantry angles. The prostate with its relatively simple geometry is treated usually with a single arc, whereas more complex anatomies (single or multiple tumors of the head and neck, for example) may require a second arc to fully treat the target volume. VMAT computations are lengthy because three large problems must be solved. First, a model of an ideal 3D dose distribution is constructed by modelling the irradiation of the target with many small X-ray beamlets subject to target dose and OAR constraints. There is no way to compute the correct distribution directly, so successively better approximations must be computed iteratively. This process is referred to as fluence map optimization, and the resulting optimal fluence map depends on the patient's anatomy and target geometries. Second, the fluence map data produced from fluence map optimization must be translated into a set of initial control points, based on the characteristics of the radiotherapy treatment machine. This process is referred to as arc sequencing. Third and finally, such control points must be optimized so that the appropriate doses indicated by the fluence map are actually accomplished by the radiotherapy treatment machine. This process is referred to as direct aperture optimization.

FIG. 8 illustrates a data flow through these three typical stages of VMAT plan development: Fluence map optimization (FMO) 820, arc sequencing 840, and direct aperture optimization 860. As shown in FIG. 8, patient image structures 810, such as image data received from CT, MRI, or similar imaging modalities, are received as input for treatment planning. Through a process of FMO 820, a fluence maps 830 are identified and created. For VMAT plans, the fluence maps 830 represent the ideal target dose coverage that must be replicated by constructing segments (MLC apertures and monitor unit weights) at a set of linac gantry angles.

Specifically, in conventional stages of VMAT planning, the fluence maps 830 provide a model of an ideal 3D dose distribution for a radiotherapy treatment, constructed during FMO 820. FMO is a hierarchical, multicriteria, numerical optimization that models the irradiation of the target with many small X-ray beamlets subject to target dose and OAR constraints. The resulting fluence maps 830 represent 2D arrays of beamlets' weights that map the radiation onto a beam's-eye-view of the target; thus, in planning a VMAT treatment, there is a fluence map for each VMAT beam at every one of the 100 or more angle settings of the linac gantry encircling the patient. Since fluence is the density of rays traversing a unit surface normal to the beam direction, and dose is the energy released in the irradiated material, the resulting 3D dose covering the target is specified by the set of 2D fluence maps.

The 3D dose that is represented in a fluence map 830, produced from FMO 820, does not include sufficient information about how a machine can deliver radiation to achieve that distribution. Therefore, an initial set of linac/MLC weighted apertures (one set per gantry angle; also called a control point) must be created by iterative modelling of the 3D dose by a succession of MLC apertures at varying gantry angles and with appropriate intensities or weights. These initial control points 850 are produced from arc sequencing 840, with the resulting apertures and parameters of (initial control points 850) being dependent on the specific patient's anatomy and target geometries.

Even with the generation of many control points 850, additional refinement of the apertures and weights is often involved, occasionally adding or subtracting a control point. Refinement is necessary since the 3D dose distribution resulting from arc sequencing 840 is degraded with respect to the original optimal fluence map 830, and some refinement of the apertures invariably improves the resulting plan quality. The process of optimizing the apertures of these control points is referred to as direct aperture optimization 860, with the resulting refined apertures and weights (final control points 870) being dependent on the specific patient's anatomy and target geometries.

In each of the operations 820, 840, 860, an achievable solution corresponds to the minimum value of an objective function in a high-dimensional space that may have many minima and requires lengthy numerical optimizations. In each case, the objective function describes a mapping or relationship between the patient's anatomic structures and a dose distribution or set of linac/MLC machine parameters.

When developing radiotherapy treatment plans for VMAT treatments, the processes of FIG. 8 are often performed in a computer using brute-force numerical optimization. The resulting plan computations for such treatments are time consuming—30 to 60 minutes for typical prostate cases and much longer for more complex head/neck treatments. Such computation time is impractical for adaptive treatments requiring plan recomputation to account for changes in patient anatomy from one treatment fraction to the next. Additionally, in VMAT planning, the objective functions of the differences between control point-dose and the optimal fluence-dose depend on thousands of variables related to the numbers of beamlets populating the trial apertures and their weights. Minimizing such a high-dimensional function with many possible local minima is a challenge, but in addition, apertures may be added or deleted during direct aperture optimization 860, and heuristic strategies for avoiding local minima may require extra processing. Minimization over small sets of nearby beamlets must be periodically interrupted to recalculate the full dose for the current set of apertures. This is itself a significant computation but is needed to update the full objective function.

The following techniques discuss a mechanism by which the generation of initial control points 850 and the process of arc sequencing 840 (and, the resulting direct aperture optimization 860) can itself be optimized from modeling. Specifically, the optimization of control points may occur through the generation of control point data using a probabilistic model, such as with a model that is trained via machine learning techniques.

In the following examples, projection reformatting is used to represent anatomy and aperture information for training and use with a model. In another set of examples, fluence map data is used for training and use with a model. Either form of this information may be input into a trained model to derive initial control points (such as are conventionally produced from arc sequencing) or a refined set of control points apertures (such as are conventionally produced from direct aperture optimization). Control points are typically represented as control point numbers (weights), whereas apertures can be are represented graphically relative to target. Because arc sequencing and direct aperture optimization both produce sets of apertures and weights, one approximate and one refined, it is possible to create machine-learned control points that accomplish faster and more uniformly accurate VMAT treatment plans.

Probabilistic modeling of control points, based on a model that is learned from populations of clinical plans, can provide two significant benefits to the control point operations discussed with reference to FIG. 8. One benefit from use of a probabilistic model is to accelerate the search for a solution. Using a trained probabilistic model, a new patient's structures can be used to infer control points (e.g., initial control points 850) that approximates a true solution. This approximation of the solution can serve as a starting point for the numerical optimization and lead to a correct solution (e.g., the final control points 870) in less time than starting from a point with less information. Another benefit from use of a probabilistic model involves the use of approximations to reliably achieve higher quality results than would be obtained by starting with less information. For instance, in some settings, the control points inferred from a machine learning model can serve as a lower bound on the expected plan quality of control point optimization.

In various examples, generative machine learning models are adapted to generate control points used as part of a radiotherapy treatment plan development. As indicated above, this may be used to shorten the plan/re-plan time for the arc sequencing and the direct aperture optimization steps used during design of a treatment plan. Both steps produce a set of machine parameters—gantry angle ϕ, apertures as sets of left and right leaf edge settings ( . . . Lₙ^{ϕ}, Rₙ^{ϕ} . . . )^{T}, and aperture cumulative monitor units y^{ϕ}—that are collectively the parameters that drive the linac and MLC to produce the actual treatment. This parameter set is also the actual treatment plan. Accurate prediction of these parameters provides the means to dispense with arc sequencing altogether and to considerably shorten the direct aperture optimization time. This occurs because the aperture optimization begins at a point much closer to the final solution for that patient than default starting points provided by commercial treatment planning systems.

As a more detailed overview, the following outlines a VMAT radiotherapy planning process, implemented with probabilistic machine learning models, for producing control point values. The following approaches may be used to generate radiotherapy plan parameters for control points given only a new patient's images and anatomy structures including OARs and treatment targets. The generation of plan parameters is made using probabilistic models of plans learned from populations of existing clinical plans. The new patient data combined with the model enables a prediction of a plan that serves as the starting point for direct aperture optimization, allowing an overall reduction in the time to develop and refine the plan to clinical quality.

The probabilistic models are built as follows. Let us represent the anatomy data as a kind of random variable X, and the plan information as random variable Y. Bayes' Rule states that the probability of predicting a plan Y given a patient X, p(Y|X), is proportional to the conditional probability of observing patient X given the training plans, Y, p(X|Y) and the prior probability of the training plans p(Y), or

$$p ( Y \vert X ) ∝ p ( X \vert Y ) p ( Y ) \qquad ( Equation 1 )$$

Bayesian inference predicts a plan Y* for a novel patient X* where the conditional probability p(Y*|X*) is drawn from the training posterior distribution p(Y|X). In practice, the novel anatomy X* is input to the trained network that then generates an estimate of the predicted plan Y* from the stored model p(Y|X).

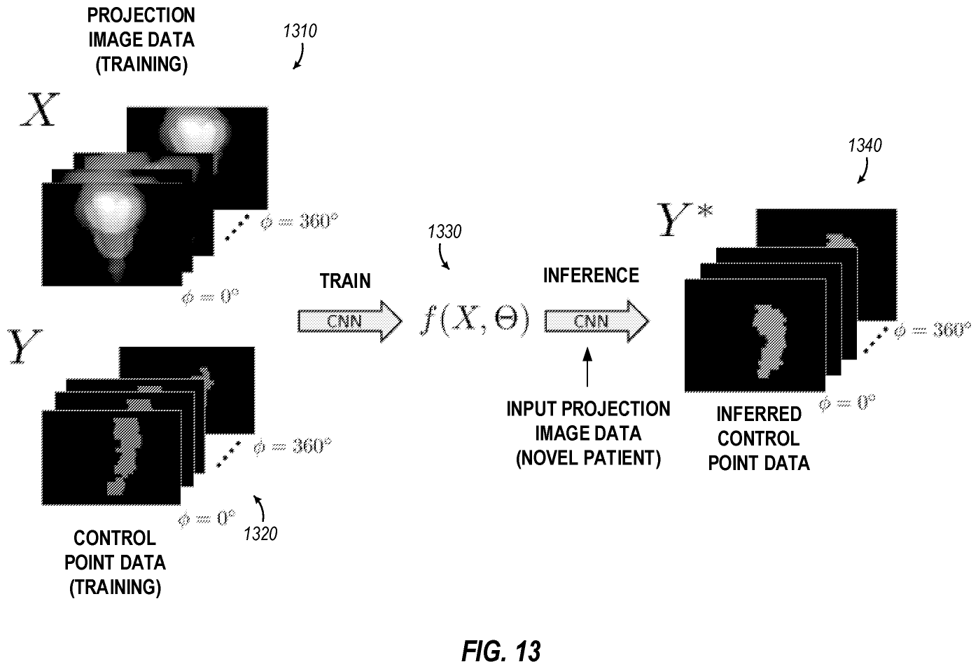

The plan posterior models p(Y|X) are built by training convolutional neural networks with pairs of known data (anatomy, plan; X, Y) in an optimization that minimizes network loss functions and simultaneously determines the values of the network layer parameters Θ. These network parameter values parameterize the posterior model, written as p(Y|X; Θ) or as the function Y=f(X, Θ) as shown in FIG. 13. Once trained, the network can infer a plan for a new anatomy by the Bayes analysis described above. Network performance is established by comparing the inferred plans for test patients not used for training with those same test patients' original clinical plans—the better the network the smaller the differences between the sets of plans.

Because the anatomy data exists in rectilinear arrays and the plan data are tuples of scalar angles and weights and lists of MLC leaf edge positions, both kinds of data must be transformed to a common coordinate frame. The anatomy images and structure contours are transformed to a cylindrical coordinate system and represented as beam's-eye-view projections of the patient volume containing the target and the nearby OARs. The MLC apertures are represented as graphic images occupying the same coordinate frame as the anatomy projections, aligned and scaled to be precisely in register with the projections. That is, at each gantry angle ϕ, one projection of the target and the corresponding aperture image are superimposed at the central axis of the cylindrical coordinate system. These transformations are described in further detail below.

FIG. 9 illustrates examples of control point aperture calculation operations, providing a comparison of a conventional control point generation process (e.g., through arc sequencing) to a machine-learning-modeled control point optimization performed with the various examples discussed herein. As shown, conventional arc sequencing iterates through aperture selection and profile optimization. With arc sequencing, a new aperture is added sequentially as the whole plan (all the objectives and constraints) is optimized. Then, the next aperture is added and the whole plan is optimized, and so on, until sufficient apertures have been defined to provide needed target coverage. Based on fluence data 901, each iteration begins with the selection of an aperture 910 added to the plan followed by multi-criterial aperture profile optimization 920 to control dosage delivered by the new beam and all previously-selected beams. The aperture value with the best score, representing a best control value, is added to the plan 940. The first optimization stage is complete when different aperture settings, identified in a search for a new direction 930, fail to improve the optimization score sufficiently. A second optimization stage 960 (direct aperture optimization) is performed to improve the objectives further if possible. The result of this iterative build-up of aperture settings and profiles is a plan 980 that is pareto-optimal with respect to the wishlist objectives and constraints.

In contrast, the machine learning-modeled control point calculation techniques discussed below begin with an estimate of aperture profiles 950, produced from image data 902 (or optionally, fluence data 904 produced from image data 902), using a model learned from a population of clinical plans. This “plan estimate” directly goes to the second optimization stage 960 (e.g., direct aperture optimization) for refinement with respect to the wishlist objectives. This avoids the time-consuming buildup of searching performed by the first optimization stage and achieves shorter times to plan creation, because the machine learning estimate starts closer to the pareto optimum in parameter space than the conventional control point parameters.

Fluence data 904 may be used as input data for machine learning modeling, because VMAT control points are dependent on the optimal fluence map. The fluence map is solved by fluence map optimization (FMO), which involves modeling the dose in tissue applied by a constellation of X-ray beamlets projected into the patient's target volume, subject to dose constraints for both the target and nearby organs-at-risk. The resulting fluence map is a 3D array of real numbers equal to the dose at each given volume element in the patient. Accordingly, the fluence map and its beamlet array (and optimization constraints) are equivalent forms of the fluence solution. It will be understood that fluences and fluence beamlets do not provide direct information about machine operation parameters. However, since fluence data 904 provides another picture of the treatment plan, such fluence information could provide additional and different information to improve the control point prediction.

There are several approaches which can be used for combining information from fluence/fluence beamlets and the apertures of control points. In a first approach, a single model may use learning for predicting control points from a combination of anatomy and fluence. This may include resampling a 3D fluence map by projections (such as described in U.S. patent application Ser. No. 16/948,486, titled “MACHINE LEARNING OPTIMIZATION OF FLUENCE MAPS FOR RADIOTHERAPY TREATMENT”, which is incorporated in reference herein in its entirety), and using machine learning for predicting control points from the combination of fluence and anatomy projections. This approach, however, may be difficult since the fluence projections may have little texture or structure (unlike the anatomy) to be encoded by a CNN.

In a second approach, two models may use learning—a first model used for the fluences predicted by anatomy and the second model used for control points predicted by anatomy. The models could be combined (as a weighted sum of layer biases and weights, for example) with the expectation that the contribution of the fluence model would improve the control point model. In a third approach, two models may use learning—a first model used for the prediction of control points from anatomy, and the second model used for the prediction of control points from fluence beamlet arrays. The models could be combined (as in the second approach) and may provide improved control point prediction by incorporating the fluence beamlet information.

Optimizing fluence distributions, and the FMO problem, can be considered according to the following. As suggested above, in IMRT and VMAT treatments, multiple (possibly many) beams are directed toward the target, and each beam's cross-sectional shape conforms to the view of the target from that direction, or to a set of segments that all together provide a variable or modulated intensity pattern. Each beam is discretized into beamlets occupying the elements of a virtual rectangular grid in a plane normal to the beam. The dose is a linear function of beamlet intensities or fluence, as expressed with the following equation:

$${d}_{i} ( b ) = {\sum }_{j = 1}^{n} {D}_{ij} {b}_{j} \qquad ( Equation 2 )$$

Where dᵢ(b) is the dose deposited in voxel i from beamlet j with intensity bⱼ, and the vector of n beamlet weights is b=(b₁ . . . , b_{B})^{T}. Dᵢⱼ; is the dose deposition matrix.

The FMO problem has been solved by multicriteria optimization. Romeijn et al. (2004) provides the following FMO model formulation:

$$( P ) : {min}_{x \geq 0} F ( d ( b ) ) \qquad ( Equation 3 )$$

$$s . t . {min}_{x \geq 0} {G}_{1} ( b ) \leq {C}_{1} ( b )$$

$$…$$

$$s . t . {min}_{x \geq 0} {G}_{L} ( b ) \leq {C}_{L} ( b )$$

where F(d(b)) is a dose objective function, whose minimization is subject to the listed constraints and where the specialized objectives G(b) are subject to dose constraints C(b), and L is the number of constraints. The objective F(d(b)) minimizes the difference of the dose being calculated d(b) with the prescribed dose P(b):

$$F ( d ( b ) ) = {\sum }_{i} {‖ {d}_{i} ( b ) - {P}_{i} ( b ) ‖}_{2}^{2} \qquad ( Equation 4 )$$

where the sum is over all voxels. Solutions of constrained objectives may be achieved several ways. Pareto optimal solutions of multicriteria problems can be generated that have the property that improving any criterion value is only possible if the at least one other criterion value deteriorates, and that all the members of a Pareto optimal family of solutions lie on a Pareto boundary in solution space. By varying the relative weights of the constraints, the planner can move along through the optimal plans to explore the effects of the target dose v, organ sparing trade-offs. An alternative solution to the FMO problem that also uses this objective and constraints is the method of Lagrange multipliers.

The individual constraints are either target constraints of the sort, “95% of target l shall receive no less than 95% of the prescription dose” or “98% of target l shall receive the prescribed dose,” or “the maximum allowed dose within target l is 107% of the prescribed dose to a volume of at least 0.03 cc.” Critical structures for which dose is to be limited are described by constraints of the sort “no more than 15% volume of structure l shall exceed 80 Gy” or “mean dose to structure l will be less than or equal to 52 Gy.” In sum, the target objectives are maximized, the critical structure constraints are minimized (structure doses are less than the constraint doses), and the beamlet weights are all greater than or equal to zero.

In practice the target and critical structure constraints often are in conflict since the target dose penumbra due to scatter frequently overlaps with nearby critical structures. Planning that involves the iterative adjustment of the constraint weights to produce desired 3D dose distributions can produce nonintuitive results and require significant planner time and effort since each weight adjustment must be followed by a re-solution for the optimal dose distribution of Equation 4.

The FMO problem to be solved for VMAT is similar to IMRT, except the beamlets are arranged in many more beams around the patient. The VMAT treatment is delivered by continuously moving the gantry around the patient, and with continuously-moving MLC leaves that reshape the aperture and vary the intensity pattern of the aperture. VMAT treatments can be delivered faster and with fewer monitor units (total beam on-time) than IMRT treatments for the same tumor. Because of the larger number of effective beams, VMAT is potentially more accurate in target coverage and organ sparing than the equivalent IMRT treatment. Further, the optimal fluence map is only the intermediate result in IMRT/VMAT planning. From the 3D fluence map, a 3D dose distribution is computed that must satisfy the gantry- and MLC leaf-motion constraints to produce a dose map that differs as little as possible from the fluence map. This is the segmentation part of the planning process and is also a constrained optimization problem.

Arc sequencing and direct aperture optimization, however, can be considered with the following. The goal of IMRT or VMAT planning is to define a set of machine parameters that instruct the linear accelerator and jaws/MLC to irradiate the patient to produce the desired dose distribution. For dynamic IMRT delivery or VMAT delivery, this includes gantry angles, or angle intervals (sectors), and the aperture(s) at each angle, and the X-ray fluence for each aperture. Dynamic deliveries mean that the gantry is rotating, and the MLC leaves are translating continuously while the beam is on. Because the beam is on and the gantry is in continuous motion, the treatment times are shorter than for static IMRT treatments.

Communication between the treatment planning system and the linac is through DICOM definitions of the machine parameters. These are: gantry angle ϕ, leaf edge positions, and the number of monitor units (MU) to be delivered for this control point. Assuming one aperture per gantry angle ϕ, the set of control points can be expressed as the ϕ-indexed sets of quantities:

$$\{ ϕ , {( … {L}_{n}^{ϕ} , {R}_{n}^{ϕ} … )}^{T} , {y}^{ϕ} \} \qquad ( Equation 5 )$$

Arc sequencing produces an initial set of aperture: shapes and weights. Methods include graph algorithms in which apertures are selected according to a minimum-distance path through a space of leaf configurations, and other more heuristic methods. The aperture shapes and weights that must be refined by direct aperture optimization (DAO). To solve for optimal apertures, one must determine for each control point the left and right leaf positions Lₙ^{ϕ}, Rₙ^{ϕ} for each n-th leaf pair, and the aperture weight or radiation intensity y^{ϕ} for gantry angle ϕ. With these parameter values, the software controlling the linac gantry and MLC can generate the sequence of apertures to deliver the planned dose distribution D(b). Analogous to Equation 3, the optimal-dose problem can be formed in terms of machine parameters:

$$\begin{matrix}{min}_{y , L , R} F ( d ) \\ s . t . {d}_{i} = {\sum }_{ϕ} {\sum }_{n} {\sum }_{j} {D}_{ijn}^{ϕ} {b}_{jn}^{ϕ} \\ where {b}_{jn}^{ϕ} = {y}^{ϕ} [ {z}_{j} ( {R}_{n}^{ϕ} ) - {z}_{j} ( {L}_{n}^{ϕ} ) ] , {y}^{ϕ} \geq 0 , {L}_{n}^{ϕ} \leq {R}_{n}^{ϕ}\end{matrix} \qquad ( Equation 6 )$$

Like Equation 4, the dose at voxel i is the summed over the contributions of many beamlets, bⱼₙ^{ϕ}, arranged at gantry angles ϕ, with MLC leaf-pairs n and leaf positions j. The beamlet intensity at angle ϕ is a function of the corresponding left and right leaf positions, Lₙ^{ϕ}, Rₙ^{ϕ}, accounting for fractional beamlets. Additionally, the beamlet intensity function y^{ϕ} must be positive semidefinite, and the left leaf edge position is always less than or equal to the corresponding right leaf edge in the coordinate system defined for the MLC.

A solution for VMAT is analogous to IMRT. The objective function of the dose, F(D(b)), is minimized with respect to the control point parameters, using gradient descent methods where the objective function-parameter derivatives are of the sort:

$$\frac{\partial F}{\partial {L}_{n}} = {\sum }_{v ∈ V} \frac{\partial F}{\partial {d}_{v}} \frac{\partial {d}_{v}}{\partial {L}_{n}} \qquad ( Equation 7 )$$

and must be evaluated over all patient voxels v that are affected by, in this example, leaf edge Lₙ. This is a more complicated optimization problem than that for IMRT where naïve application of the gradient minimization of Equation 5 would be computationally prohibitive. This implies a necessarily sparse solution with stringent regularity conditions as well.

The following provides details for a training embodiment to learn machine parameters (control points) from a set of patient treatment plans. A challenge for control point prediction based on anatomy is that the anatomy and control points have fundamentally different common representations. Anatomies are depicted by rectilinear medical images of various modalities and control points are vectors of real number parameters. Further, even if the control points' apertures are represented by a graphical representation in an image, the orientation of the aperture does not correspond to any of the standard 2D or 3D views of anatomy. As the linac travels in an arc around the patient, the anatomy view at any moment is a projection image of the anatomy, equivalent to a plane radiograph of that anatomy at that angle. Therefore, using projections of patient anatomy requires that control point aperture data be reformatted and aligned with the anatomy projections at the corresponding angles.

FIG. 10 depicts the creation of multiple anatomy projection images 1010, 1020, 1030 from a 3D volume of CT image data. An equivalent technique can be used to produce projections for MR images, and thus it will be understood that the following references to CT image data is provided for purposes of illustration and not limitation.

As depicted in FIG. 10, multiple projections of the male pelvic organs are depicted relative to a 3D CT image 1001 of that anatomy, provided with views 1010, 1020, 1030 at 0, 45, and 90 degrees respectively (introduced earlier with respect to FIG. 2A). The patient orientation is head-first supine with the head of the patient beyond the top of the projections. The organs at risk (bladder, rectum), the target organs (prostate, seminal vesicles), and their encapsulating target volumes (Target1, Target2) are delineated (contoured) and each organ voxel was assigned a constant density value, and densities were summed for voxels in two or more structures.

Projection images through this anatomy about the central axis of the 3D CT volume 1000 and at the assigned densities may be obtained, for example, using a forward projection capability of the RTK cone beam CT reconstruction toolkit, an open-source cone-beam CT reconstruction toolkit based on the Insight Toolkit (ITK). In these views, the bladder at 0° is in front of the seminal vesicles (bladder is closest to the viewer) and rotates to the left in the next two views. Projection images and their variants-digitally reconstructed radiographs and beam's eye views—are important in radiation therapy, providing checks on the co-location of the target and the beam shape and for quantitation of beam dose across the target. Projections can be computed either by directly re-creating the projection view geometry by ray tracing or by Fourier reconstruction as in computed tomography.

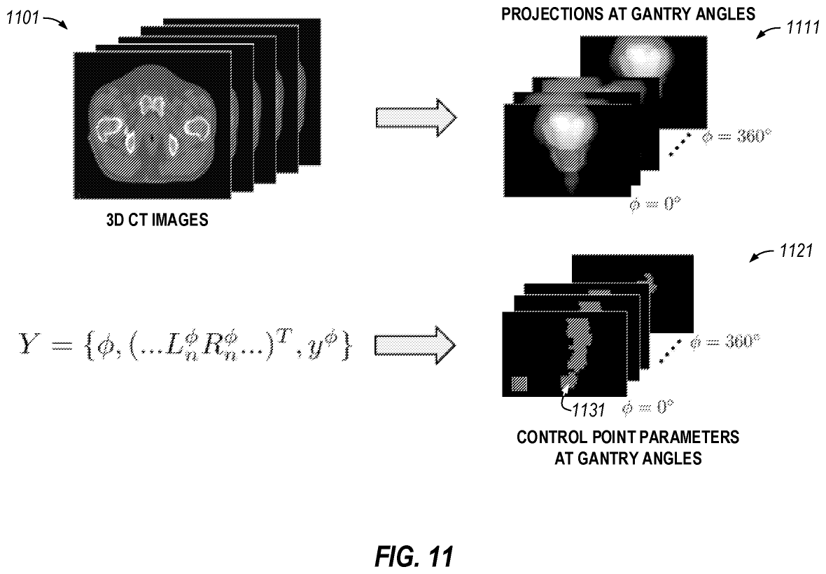

FIG. 11 depicts transformations of images and control point parameters into 3D image volumes, corresponding to the volume depicted in FIG. 10. Here, the top row demonstrates the recreation of 3D CT image data 1101 as a stack of projections 1111 taken at a set of gantry angles. The control point apertures represented by left and right leaf edge positions ( . . . Lₙ^{ϕ}Rₙ^{ϕ} . . . )^{T} fare recreated as graphical images 1121 (bottom row), illustrating the openings (e.g., opening 131) between ML C left and right leaf edges that permit radiation to pass. These images are aligned and scaled with the projections such that each projection pixel is aligned with the corresponding aperture pixel that irradiates it.

The control point parameters represent the gantry angles, the MLC apertures at each gantry angle (gaps between left and right MLC leaf edges), and the radiation intensity at that angle. In FIG. 11, the apertures are depicted as graphical images, with the assignment of one aperture image to each anatomy projection image at the same gantry angle, Each image element (e.g., the element represented by 1131) represents an opening between pairs of opposing tungsten leaves, and it is these apertures that shape the X-ray beam to cover the target to the prescribed radiation dose. The projections and the apertures are scaled and aligned to ensure that each anatomy pixel is aligned with the corresponding aperture pixel irradiating that anatomy element. That construction of anatomy and control point data are represented as aligned 3D image volumes with common dimensions, pixel spacing, and origin.

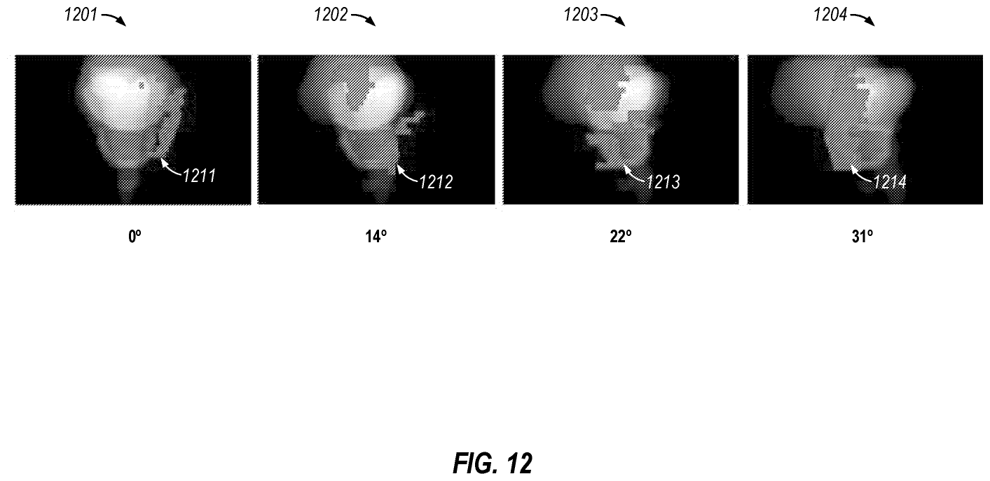

FIG. 12 depicts a superposition of projected anatomy and control point apertures for a 30° arc interval, in images 1201, 1202, 1203, 1204. Note that both the apertures and the projections depict the motions of the treatment machine (revolving patient) and the MLC leaves (right-to-left sweep of the features 1211, 1212, 1213, 1214).

In more detail, FIG. 12 depicts a superposition of projections of the pelvic organs with MLC apertures (represented by features 1211, 1212, 1213, 1214) for several angles from 0° to about 30°. The apertures are the openings between left and right banks of tungsten leaves permitting X-ray radiation to reach the target, as the target revolves in the view of the linac MLC. The 0° view is the same as that in FIG. 11. The MLC apertures are designed by the treatment planning program to irradiate the target volume to the prescribed dose. For this 30° interval, the MLC aperture sweeps from right to left while the anatomy revolves under it. A full 360° arc corresponds to six back-and-forth sweeps of the apertures. While all the organs in this view are irradiated, most of the accumulated dose is confined to a target volume including the prostate and the seminal vesicles.

FIG. 13 depicts a schematic of the deep learning procedure to train a model to predict control point parameters, within a data environment of a CNN network. The learned model enables the inference of the estimated aperture data Y* that is then translated into a “synthetic” DICOM RT Plan and input into a treatment planning program for analysis. Because the control point parameters dictate the action of the linac and MLC treatment delivery, prediction of the control points is equivalent to predicting the treatment plan.

In the schematic of FIG. 13, the training data are pairs of 3D projections 1310 and 3D stacks of control point representations (apertures) 1320 from the same patient. Training produces the model f 1330 from which an estimate Y* can be inferred. The estimate is itself a 3D data volume 1340 with the same size and shape as the input anatomy and aperture data volumes. The estimate can be translated into a functional set of control points and used as a warm start to accelerate direct aperture optimization. Further, the estimate may be translated into a DICOM RT Plan file and input to a treatment planning program for comparison with a ground truth plan. Because the control point parameters dictate the action of the linac and MLC treatment delivery, prediction of the control points is equivalent to predicting the treatment plan itself.

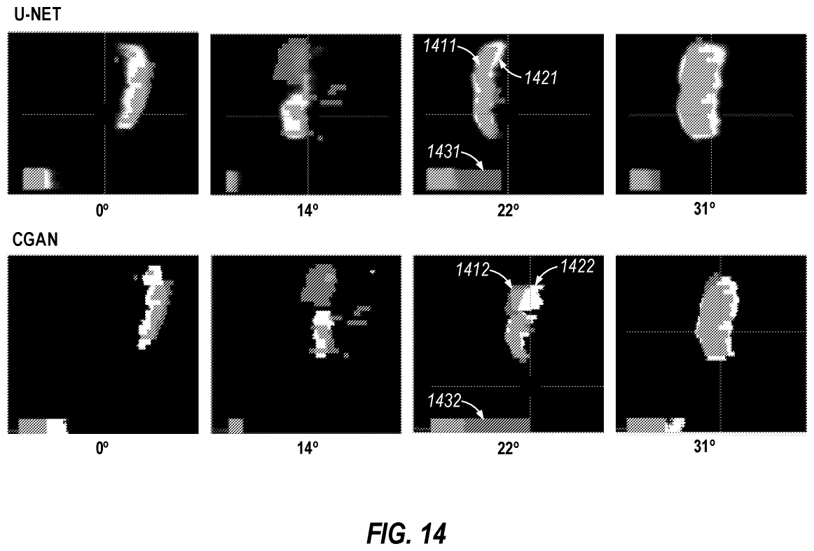

FIG. 14 displays results of training on a set of prostate treatment plans by two different CNNs—3D U-Net and a 3D conditional GAN. The aperture shapes in each frame (e.g., aperture shapes 1411, 1412) are the ground truth MLC apertures at the indicated gantry angles and superimposed to the CNN estimated apertures (e.g., aperture shapes 1421, 1422). The estimated beam intensities are represented by the lengths of the bars (bars 1431, 1432) in the lower-left of each figure. Qualitatively the agreement of test aperture (shapes 1421, 1422) with the ground truth aperture (shapes 1411 and 1412) is about the same for the U-Net and the cGAN.

The approximate control points may be refined by the segment shape and weight optimization functionality of a treatment planning program to make them suitable for clinical use. As will be understood, CNN estimates for control points that are as close as possible to the ground truth plan control points will take less time to optimize to produce a clinically usable plan.

In an example, learning of treatment machine parameters from a population of patient treatment plans may occur with the following configuration of a CNN. Here, CNNs are trained to determine the relationship between observed data X and target domain Y. The data X is a collection of 3D planning CTs, anatomy voxel label maps, and functions of the labelled objects' distances from one another. The target Y is a set of K control points defining the machine delivery of the treatment,

$$y = {\{ ϕ , {( … {L}_{n}^{ϕ} , {R}_{n}^{ϕ} … )}^{T} , {y}^{ϕ} \}}_{k} , k = 1 , … , K \qquad ( Equation 8 )$$

The action of the CNN is symbolized by the function f(·)

$${Y}^{*} = f ( X ; Θ ) \qquad ( Equation 9 )$$

where Θ=(θ₁, . . . , θₙ)^{T} is a vector of the neural net parameters for which the Y* is the closest approximation of the true Y. The CNN is trained using paired data sets {X, Y}ᵢ, i=1, . . . , N of images, anatomy labels, or other anatomy representations (e.g., signed distance maps) X and known control points Y. Training minimizes a cost function [symbol] (Θ) such as:

$$ℒ ( {Θ}^{*} ) = {arg min}_{Θ} [ ‖ f ( X ; Θ ) - \hat{Y} {‖}_{K} + λ {‖ Θ ‖}_{L} ] , K , L ∈ \{ 1 , 2 \} \qquad ( Equation 10 )$$

where Θ* is the set of parameters the minimizes the mean squared error between the true Y and the estimate Y*. In deep learning the cost functions frequently express the data approximation function as the conditional likelihood of observing Y given X subject to the values of the parameters Θ, expressed as P(X|Y; Θ). The optimal parameters Θ_{OPT} are obtained by maximizing the likelihood, or training the CNN,

$${Θ}_{O P T} = arg {max}_{Θ} P ( Y \vert X ; Θ ) \qquad ( Equation 11 )$$

or alternatively,

$${Θ}_{O P T} = arg {max}_{Θ} {\sum }_{t ∈ T} P ( {Y}_{t} \vert {X}_{t} ; Θ ) \qquad ( Equation 12 )$$

summed over the training data T.

For CNN programs designed to learn information from images, the control point data might be presented to the network in the form of images with fixed formats specifying the apertures, angles and intensity values. Alternatively, the input patient images might be pooled with the control point parameters presented as real arrays. Other forms or data presentation might be applicable as well. Because the control point parameters dictate the action of the linac and MLC treatment delivery, prediction of the control points is equivalent to predicting the treatment plan itself.

In various examples, various forms of machine learning models may be implemented by artificial neural networks (NNs). At its simplest implementation, a NN consists of an input layer, a middle or hidden layer, and an output layer. Each layer consists of nodes that connect to more than one input node and connect to one or more output nodes. Each node outputs a function of the sum of its inputs x=(x₁, . . . , xₙ), y˜ϕ(w^{T}x+β), where w is the vector of input node weights and β is the layer bias and the nonlinear function σ is typically a sigmoidal function. The parameters Θ=(w, β) are the realization of the model learned to represent the relationship Y=f(X; Θ). The number of input layer nodes typically equals the number of features for each of a set of objects being sorted into classes, and the number of output layer nodes is equal to the number of classes. For regression, the output layer typically has a single node that communicates the estimated or probable value of the parameter.

A network is trained by presenting it with object features where the object's class or parameter value is known and adjusting the node weights w and biases β to reduce the training error by working backward from the output layer to the input layer—an algorithm called backpropagation. The training error is a normed difference ∥y−f(x)∥ between the true answer y and the inference estimate f(x) at any stage of training. The trained network then performs inference (either classification or regression) by passing data forward from input to output layer, computing the nodal outputs σ(w^{T}x+β) at each layer.

Neural networks have the capacity to discover general relationships between the data and classes or regression values, including non-linear functions with arbitrary complexity. This is relevant to the problem of radiotherapy dose prediction, or treatment machine parameter prediction, or plan modelling, since the shape or volume overlap relationships of targets and organs as captured in the dose-volume histogram and the overlap-volume histogram are highly non-linear and have been shown to be associated with dose distribution shape and plan quality.

Modern deep convolutional neural networks (CNNs) have many more layers (are much deeper) than early NNs—and may include dozens or hundreds of layers, each layer composed of thousands to hundreds of thousands of nodes, with the layers arranged in complex geometries. In addition, the convolution layers map isomorphically to images or any other data that can be represented as multi-dimensional arrays and can learn features embedded in the data without any prior specification or feature design. For example, convolution layers can locate edges in pictures, or temporal/pitch features in sound streams, and succeeding layers find larger structures composed of these primitives. In the past half-dozen years, some CNNs have approached human performance levels on canonical image classification tests-correctly classifying pictures into thousands of classes from a database of millions of images.

CNNs are trained to learn general mappings f: X→Y between data in source and target domains X, Y, respectively. Examples of X include images of patient anatomy or functions of anatomy conveying structural information. Examples of Y could include maps of radiation fluence or delivered dose, or maps of machine parameters superposed onto the target anatomy X. As indicated in FIG. 14, pairs of matched, known X, Y data may be used to train a CNN. The CNN learns a mapping or function f(X; Θ) of both anatomy and network parameters Θ=(Θ₁, . . . , Θₙ)^{T} where Θᵢ={wᵢ, βᵢ} are the parameters for the i-th layer. As suggested above, training minimizes a loss function [symbol] (Θ) over the mapping f and a ground truth or reference plan parameter Ŷ

$$ℒ ( {Θ}^{*} ) = {arg min}_{Θ} [ {‖ f ( X ; Θ ) - \hat{Y} ‖}_{K} + λ {‖ Θ ‖}_{L} ] , K , L ∈ \{ 1 , 2 \} \qquad ( Equation 13 )$$

where the first term minimizes the difference between the network estimated target f(X; Θ) and the reference property Ŷ and the second term minimizes the variation of the values of the Θ. Subscripts K, L specify the norm. The L2 norm (K, L=2) is globally convex but produces blurred estimates of Y while the L1 norm (K, L=1) encourage sharper estimates. Network performance typically dictates what combination of norms are useful.

FIG. 15A depicts a schematic of a U-Net deep convolutional neural network (CNN). Specifically, this schematic depicts the U-Net deep CNN model adapted for generating estimated control point representations (images) from a generative arrangement, such as to provide a generative model adapted for the techniques discussed herein. Shown are a pair of input images representing target anatomy constraints (top image) and a radiotherapy treatment control point representation corresponding to that target anatomy (bottom image), provided in an input training set 1510 to train the network. The output is a predicted control point representation 1540, inferred for a target image. The input training set 1510 may include individual pairs of input images that are projected from a 3D anatomy imaging volume and 3D control point image volume; these individual pairs of input images may comprise individual images that are projected at relevant beam angle used for treatment with a radiotherapy machine. The output data set, provided in the control point representation 1540, is a representation that may comprise individual output images or a 3D image volume.

A U-Net CNN creates scaled versions of the input data arrays on the encoding side by max pooling and re-combines the scaled data with learned features at increasing scales by transposed convolution on the encoding side to achieve high performance inference. The black rectangular blocks represent combinations of convolution/batch normalization/rectified linear unit (ReLU) layers; two or more are used at each scale level. The blocks' vertical dimension corresponds to the image scale (S) and the horizontal dimension is proportional to the number of convolution filters (F) at that scale. Equation 13 above is a typical U-Net loss function.

The model shown in FIG. 15A depicts an arrangement adapted for generating an output data set (output control point representation images 1540) based on an input training set 1510 (e.g., paired anatomy images and control point representation images). The name derives from the “U” configuration, and as is well understood, this form of CNN model can produce pixel-wise classification or regression results. In some cases, a first path leading to the CNN model includes one or more deformable offset layers and one or more convolution layers including convolution, batch normalization, and an activation such as the rectified linear unit (ReLU) or one of its variants.

The left side of the model operations (the “encoding” operations 1520) learns a set of features that the right side (the “decoding” operations 1530) uses to reconstruct an output result. The U-Net has n levels consisting of conv/BN/ReLU (convolution/batch normalization/rectified linear units) blocks 1550, and each block has a skip connection to implement residual learning. The block sizes are denoted in FIG. 15A by “S” and “F” numbers; input images are S×S in size, and the number of feature layers is equal to F. The output of each block is a pattern of feature responses in arrays the same size as the images.

Proceeding down the encoding path, the size of the blocks decreases by ½ or 2⁻¹ at each level while the size of the features by convention increases by a factor of 2. The decoding side of the network goes back up in scale from S/2″ while adding in feature content from the left side at the same level; this is the copy/concatenate data communication. The differences between the output image and the training version of that image drives the generator network weight adjustments by backpropagation. For inference, or testing, with use of the model, the input would be a single projection image or collection of multiple projection images of radiotherapy treatment constraints (e.g., at different beam or gantry angles) and the output would be graphical control point representation images 1540 (e.g., one or multiple graphical images corresponding to the different beam or gantry angles).

The representation of the model of FIG. 15A specifically illustrates the training and prediction of a generative model, which is adapted to perform regression rather than classification. FIG. 15B illustrates an exemplary CNN model adapted for discriminating a synthetic control point representation(s) from input images 1560 according to the present disclosure. As used herein, a “synthetic” image refers to a model-generated image, and thus “synthetic” is used interchangeably herein with the terms “estimated”, “predicted”, “computer-simulated”, or “computer-generated”. The discriminator network shown in FIG. 15B may include several levels of blocks configured with stride-2 convolutional layers, batch normalization layers and ReLU layers, and separated pooling layers. At the end of the network, there will be one or a few fully connection layers to form a 2D patch for discrimination purposes. The discriminator shown in FIG. 15B may be a patch-based discriminator configured to receive input synthetic control point representation images (e.g., generated from the generator shown in FIG. 15A), classify the image as real or fake, and provide the classification as output detection results 1570.

In an example, the present control point modeling techniques (e.g., used for generating VMAT control points) may be generated using a specific a type of CNN—generative adversarial networks (GANs)—that predict control point aperture parameters (control points) from new patient anatomy. The following provides an overview of relevant GAN technologies.

Generative adversarial networks are generative models (generate probability distributions) that learn a mapping from random noise vector z to output image y as G: z→y. Conditional adversarial networks learn a mapping from observed image x and random noise z as G: {x, z}→y. Both adversarial networks consist of two networks: a discriminator (D) and a generator (G). The generator G is trained to produce outputs that cannot be distinguished from “real” or actual training images by an adversarial trained discriminator D that is trained to be maximally accurate at detecting “fakes” or outputs of G.

The conditional GAN differs from the unconditional GAN in that both discriminator and generator inferences are conditioned on an example image of the type X in the discussion above. The conditional GAN loss function is expressed as:

$${ℒ}_{cGAN} ( G , D ) = {𝔼}_{x , y \sim {p}_{data} ( x , y )} [ log ( D ( x , y ) ) ] \qquad ( Equation 14 )$$

$${E}_{x \sim {p}_{data} ( x ) , z \sim {p}_{z} ( z )} [ log ( 1 - D ( x , G ( x , z ) ) ) ]$$

where G tries to minimize this loss against an adversarial D that tries to maximize it, or,

$${G}^{*} = {arg min}_{G} {max}_{D} {ℒ}_{cGAN} ( G , D ) \qquad ( Equation 15 )$$

In addition, one wants the generator G to minimize the difference between the training estimates and the actual training ground truth images,

$${ℒ}_{L 1} ( G ) = {𝔼}_{x , y \sim {p}_{data} ( x , y ) , z \sim {p}_{z} ( z )} [ {‖ y - G ( x , z ) ‖}_{1} ] \qquad ( Equation 16 )$$

so, the complete loss is the λ-weighted sum of two losses:

$${G}^{*} = {arg min}_{G} {max}_{D} {ℒ}_{cGAN} ( G , D ) + λ {ℒ}_{L 1} ( G ) \qquad ( Equation 17 )$$

In an example, the generator in the conditional GAN may be a U-Net.

Consistent with examples of the present disclosure, the treatment modeling methods, systems, devices, and/or processes based on such models include two stages: training of the generative model, with use of a discriminator/generator pair in a GAN; and prediction with the generative model, with use of a GAN-trained generator. Various examples involving a GAN and a cGAN for generating control point representation images are discussed in detail in the following examples. It will be understood that other variations and combinations of the type of deep learning model and other neural-network processing approaches may also be implemented with the present techniques. Further, although the present examples are discussed with reference to images and image data, it will be understood that the following networks and GAN may operate with use of other non-image data representations and formats.

FIG. 16 illustrates a data flow for training and use of a GAN adapted for generating control point parameters (each, a control point representation) from a received set of projection images that represents a view of an anatomy of a subject image. For instance, the generator model 1632 of FIG. 16, which is trained to produce a trained generator model 1660, may be trained to implement the processing functionality provided as part of the image processor 114 in the radiotherapy system 100 of FIG. 1.

Accordingly, a data flow of the GAN model usage 1650 (prediction or inference) is depicted in FIG. 16 as the provision of new patient data 1670 (e.g., a projection image that represents radiotherapy treatment constraints in a view of an anatomy of a subject input images from a novel patient) to a trained generator model 1660, and the use of the trained generator model 1660 to produce a prediction or estimate of a generator output (images) 1680 (e.g., control point representation images corresponding to the input projection image that represents a view of an anatomy of a subject image). A projection image can be generated from one or more CT or MR images of a patient anatomy representing a view of the anatomy from a given beam position (e.g., at an angle of the gantry) or other defined positions.

GANs comprise two networks: a generative network (e.g., generator model 1632) that is trained to perform classification or regression, and a discriminative network (e.g., discriminator model 1640) that samples the generative network's output distribution (e.g., generator output (images) 1634) or a training control point representation image from the training images 1623 and decides whether that sample is the same or different from the true test distribution. The goal for this system of networks is to drive the generator network to learn the ground truth model as accurately as possible such that the discriminator net can only determine the correct origin for generator samples with 50% chance, which reaches an equilibrium with the generator network. The discriminator can access the ground truth but the generator only accesses the training data through the response of the detector to the generator's output.

The data flow of FIG. 16 also illustrates the receipt of training input 1610, including various values of model parameters 1612 and training data 1620, with such training images 1623 including a set of projection images that represent different views of an anatomy of subject patient imaging data paired with real control point representation images corresponding to the patient imaging data at the different views, and conditions or constraints 1626. These conditions or constraints 1626 (e.g., one or more radiotherapy treatment target areas, one or more organs at risk areas, etc.) may be indicated directly in the anatomy images themselves (e.g., as shown with projection image 1010), or provided or extracted as a separate data set. The training input 1610 is provided to the GAN model training 1630 to produce a trained generator model 1660 used in the GAN model usage 1650.

As part of the GAN model training 1630, the generator model 1632 is trained on real training control point representation images and corresponding training projection images that represent views of an anatomy of a subject image pairs 1622 (also depicted in FIG. 16 as 1623), to produce and map segment pairs in the CNN. In this fashion, the generator model 1632 is trained to produce, as generator output (images) 1634, computer-simulated (estimated or synthetic) images of control point representations. The discriminator model 1640 decides whether a simulated control point representation image or images is from the training data (e.g., the training or true control point representation images) or from the generator (e.g., the estimated or synthetic control point representation images), as communicated between the generator model 1632 and the discriminator model 1640. The discriminator output 1636 is a decision of the discriminator model 1640 indicating whether the received image is a simulated image or a true image and is used to train the generator model 1632. In some cases, the generator model 1632 is trained utilizing the discriminator on the generated images. This training process results in back-propagation of weight adjustments 1638, 1642 to improve the generator model 1632 and the discriminator model 1640.

During training of generator model 1632, a batch of training data can be selected from the patient images (indicating radiotherapy treatment constraints) and expected results (control point representations). The selected training data can include at least one projection image of patient anatomy representing a view of the patient anatomy from a given beam/gantry angle and the corresponding training or real control point representations image at that given beam/gantry angle. The selected training data can include multiple projection images of patient anatomy representing views of the same patient anatomy from multiple equally spaced or non-equally spaced angles (e.g., at gantry angles, such as from 0 degrees, from 15 degrees, from 45 degrees, from 60 degrees, from 75 degrees, from 90 degrees, from 10⁵ degrees, from 120 degrees, from 135 degrees, from 150 degrees, from 165 degrees, from 180 degrees, from 195 degrees, from 210 degrees, from 225 degrees, from 240 degrees, from 255 degrees, from 270 degrees, from 285 degrees, from 300 degrees, from 315 degrees, from 330 degrees, from 345 degrees, and/or from 360 degrees) and the corresponding training control point representation image and/or machine parameter data at those different equally-spaced or non-equally spaced gantry angles.

Thus, in this example, data preparation for the GAN model training 1630 requires control point representation images that are paired with projection images that represent views of an anatomy of subject images (these may be referred to as training projection images that represent a view of an anatomy of a subject image at various beam/gantry angles). Namely, the training data includes paired sets of control point representation images at the same gantry angles as the corresponding projection images. In an example, the original data includes pairs of projection images that represents a view of an anatomy of a subject at various beam/gantry angles and corresponding control point representations at the corresponding beam/gantry angles that may be registered and resampled to a common coordinate frame to produce pairs of anatomy-derived images. The training data can include multiple of these paired images for multiple patients at any number of different beam/gantry angles. In some cases, the training data can include 360 pairs of projection images and control point representation images, one for each angle of the gantry for each training patient.

The expected results can include estimated or synthetic graphical control point representations, that can be further optimized and converted into control point parameters for generating a beam shape at the corresponding beam/gantry angle to define the delivery of radiation treatment to a patient. The control points or machine parameters can include at least one beam/gantry angle, at least one multi-leaf collimator leaf position, and at least one aperture weight or intensity.

In detail, in a GAN model, the generator (e.g., generator model 1632) learns a distribution over the data x, p_{G}(x), starting with noise input with distribution p_{Z}(z) as the generator learns a mapping G(z; θ_{G}) p_{Z}(Z)→p_{G}(x) where G is a differentiable function representing a neural network with layer weight and bias parameters BG. The discriminator, D(x; θ_{D}) (e.g., discriminator model 1640), maps the generator output to a binary scalar {true, false}, deciding true if the generator output is from actual data distribution P_{data}(x) and false if from the generator distribution P_{G}(x). That is, D(x) is the probability that X came from P_{data}(x) rather than from P_{G}(x). In another example, paired training data may be utilized in which, for instance, Y is conditioned (dependent) on X. In such cases, the GAN generator mapping is represented by G (y|x; θ_{G}): X→Y from data domain X where data x ∈X represents the anatomy projection images and domain Y where data y ∈Y represents the control point representation values corresponding to x. Here an estimate for a control point representation value is conditioned on its projection. Another difference from the straight GAN is that instead of a random noise z input, the projection image x is the generator input. For this example, the setup of the discriminator is the same as above. In general, the generator model 1632 and the discriminator model 1640 are in a circular data flow, where the results of one feed into the other. The discriminator takes either training or generated images and its output is used to both adjust the discriminator weights and to guide the training of the generator network.

In some examples, a processor (e.g., of radiotherapy system 100) may apply image registration to register real control point representation training images to a training collection of projection images. This may create a one-to-one corresponding relationship between projection images at different angles (e.g., beam angles, gantry angles, etc.) and control point representation images at each of the different angles in the training data. This relationship may be referred to as paired or a pair of projection images and control point representation images.

The preceding examples provide an example of how a GAN or a conditional GAN may be trained based on a collection of control point representation images and collection of projection image pairs, specifically from image data in 2D or 3D image slices in multiple parallel or sequential paths. It will be understood that the GAN or conditional GAN may process other forms of image data (e.g., 3D, or other multi-dimensional images) or representations of this data including in non-image format. Further, although only grayscale (including black and white) images are depicted by the accompanying drawings, it will be understood that other image formats and image data types may be generated and/or processed by the GAN.

FIG. 17 illustrates an example of a method 1700 for training a neural network model, trained for determining a control point representation such as using the techniques discussed above.

Operation 1710 includes obtaining pairs of training anatomy projection images (optionally, capturing such images), and operation 1720 includes obtaining corresponding pairs of training control point projection images (optionally, capturing such images). In an example, the following training process for the neural network model uses pairs of anatomy projection images and control point images from a plurality of human subjects, and each individual pair is provided from a same human subject.

Operation 1730 includes performing training of a model (e.g., a neural network) to configure such model to generate control point images from input anatomy projection images. In an example, the neural network model is trained with operations including: identifying multiple sets of training anatomy projection images, each set of the training anatomy projection images indicating one or more target dose areas and one or more organs-at-risk areas in the anatomy of a respective subject; identifying multiple sets of training control point images corresponding to the training anatomy projection images, each set of the training control point images indicating a control point for at a respective beam angle of the radiotherapy machine used with radiotherapy treatment of the respective subject; and training the neural network model based on the training anatomy projection images that correspond to the training control point images.

In an example, the neural network model is a generative model of a generative adversarial network (GAN) (or, a conditional adversarial generative network) comprising at least one generative model and at least one discriminative model, and the at least one generative model and the at least one discriminative model correspond to respective generative and discriminative convolutional neural networks. Specific operations applicable to training with a GAN (operations 1740-1760) include: at operation 1740, performing adversarial training to train a generative model to produce a control point image; at operation 1750, performing adversarial training to train a discriminative model to classify a generated image as synthetic or real; and at operation 1760, using adversarial training results to improve training of the generative model. Further details on GAN training is provided above.

The method 1700 concludes with operation 1770, to provide a trained generative model for use with patient anatomy projection image(s).

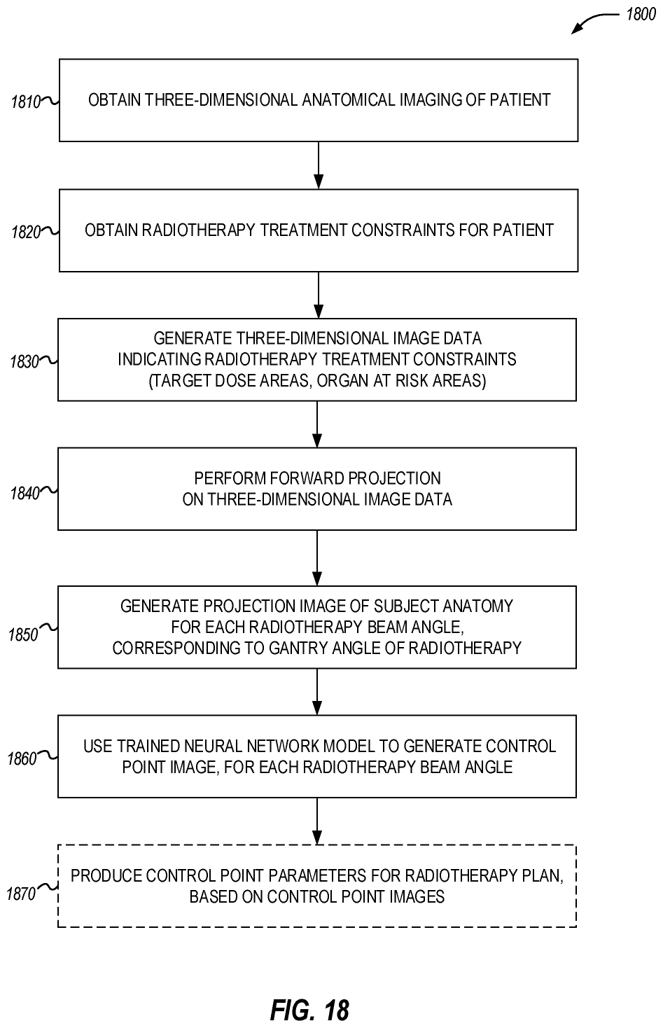

FIG. 18 illustrates an example of a method 1800 for using a trained neural network model, for determining a control point representation, based on the techniques discussed above. For instance, the trained neural network model may be provided from the results of the method 1700.

Operation 1810 includes obtaining three-dimensional anatomical imaging data (e.g., CT or MR image data) corresponding to a patient (human subject) of radiotherapy treatment, and operation 1820 includes obtaining radiotherapy treatment constraints for patient for this treatment. Such radiotherapy treatment constraints may be defined or established as part of a therapy plan, consistent with the examples of radiotherapy discussed above.

Operation 1830 includes generating three-dimensional image data which indicates the radiotherapy treatment constraints (e.g., one or more target dose areas and one or more organs-at-risk areas in anatomy of the subject) and other treatment specifications. In other examples, these treatment constraints and specifications may be provided in other data formats.

Operation 1840 includes performing forward projection on the three-dimensional image data, and operation 1850 includes generating anatomy projection images from the image data. In an example, each anatomy projection image provides a view of the subject from a respective beam angle of the radiotherapy treatment (e.g., a gantry angle).

Operation 1860 includes using a trained neural network model to generate a control point image, for each radiotherapy beam angle. In an example, each of the control point images indicates an intensity and one or more apertures of a control point of the radiotherapy treatment to apply at a respective beam angle. The neural network model may be trained with corresponding pairs of training anatomy projection images and training control point images, as described with reference to FIG. 17.

Operation 1870 includes producing control point parameters for radiotherapy plan, based on the generated control point images. For instance, this may include generating a set of final control points for use in the radiotherapy treatment to control a radiotherapy treatment machine, based on an optimization of the control points of the radiotherapy treatment indicated by the generated control point images.

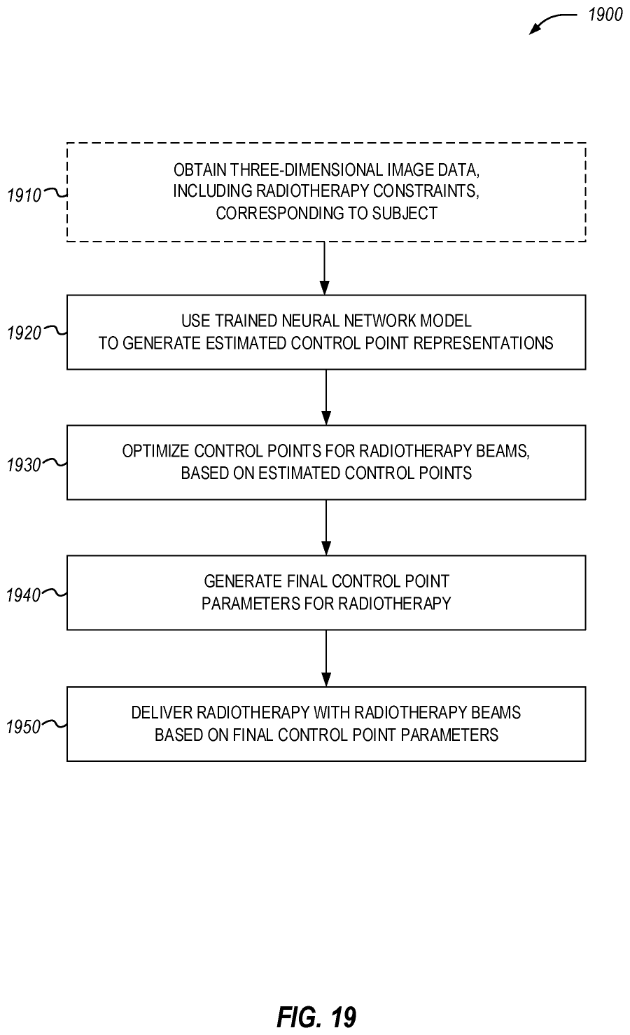

FIG. 19 is a flowchart illustrating example operations of the image processing device 112 in performing process 1900, according to example examples. The process 1900 may be embodied in computer-readable instructions for execution by one or more processors such that the operations of the process 1900 may be performed in part or in whole by the functional components of the image processing device 112; accordingly, the process 1900 is described below by way of example with reference thereto. However, in other examples, at least some of the operations of the process 1900 may be deployed on various other hardware configurations. The process 1900 is therefore not intended to be limited to the image processing device 112 and can be implemented in whole, or in part, by any other component. Some or all of the operations of process 1900 can be in parallel, out of order, or entirely omitted.

At operation 1910, image processing device 112 obtains three-dimensional image data, including radiotherapy constraints, corresponding to subject.

At operation 1920, image processing device 112 uses the trained neural network model to generate estimated control point representations.

At operation 1930, image processing device 112 optimizes the control points for the radiotherapy beams, based on the estimated control point representations.

At operation 1940, image processing device 112 generates final control point parameters for radiotherapy based on the optimized control points.

At operation 1950, image processing device 112 delivers radiotherapy with radiotherapy beams based on final control point parameters.

Further variation with the use of the trained neural network model, control point optimization, control point parameter generation, and radiotherapy delivery, may be provided with any of the examples discussed above.

FIG. 20 illustrates a block diagram of an example of a machine 2000 on which one or more of the methods as discussed herein can be implemented. In one or more examples, one or more items of the image processing device 112 can be implemented by the machine 2000. In alternative examples, the machine 2000 operates as a standalone device or may be connected (e.g., networked) to other machines. In one or more examples, the image processing device 112 can include one or more of the items of the machine 2000. In a networked deployment, the machine 2000 may operate in the capacity of a server or a client machine in server-client network environment, or as a peer machine in a peer-to-peer (or distributed) network environment. The machine may be a personal computer (PC), server, a tablet, smartphone, a web appliance, edge computing device, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example machine 2000 includes processing circuitry or processor 2002 (e.g., a CPU, a graphics processing unit (GPU), an ASIC, circuitry, such as one or more transistors, resistors, capacitors, inductors, diodes, logic gates, multiplexers, buffers, modulators, demodulators, radios (e.g., transmit or receive radios or transceivers), sensors 2021 (e.g., a transducer that converts one form of energy (e.g., light, heat, electrical, mechanical, or other energy) to another form of energy), or the like, or a combination thereof), a main memory 2004 and a static memory 2006, which communicate with each other via a bus 2008. The machine 2000 (e.g., computer system) may further include a video display device 2010 (e.g., a liquid crystal display (LCD) or a cathode ray tube (CRT)). The machine 2000 also includes an alphanumeric input device 2012 (e.g., a keyboard), a user interface (UI) navigation device 2014 (e.g., a mouse), a disk drive or mass storage unit 2016, a signal generation device 2018 (e.g., a speaker), and a network interface device 2020.

The disk drive unit 2016 includes a machine-readable medium 2022 on which is stored one or more sets of instructions and data structures (e.g., software) 2024 embodying or utilized by any one or more of the methodologies or functions described herein. The instructions 2024 may also reside, completely or at least partially, within the main memory 2004 and/or within the processor 2002 during execution thereof by the machine 2000, the main memory 2004 and the processor 2002 also constituting machine-readable media.

The machine 2000 as illustrated includes an output controller 2028. The output controller 2028 manages data flow to/from the machine 2000. The output controller 2028 is sometimes called a device controller, with software that directly interacts with the output controller 2028 being called a device driver.

While the machine-readable medium 2022 is shown in an example to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present disclosure, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), EEPROM, and flash memory devices; magnetic disks such as internal hard disks and removable disks; magneto-optical disks; and CD-ROM and DVD-ROM disks.

The instructions 2024 may further be transmitted or received over a communications network 2026 using a transmission medium. The instructions 2024 may be transmitted using the network interface device 2020 and any one of a number of well-known transfer protocols (e.g., HTTP). Examples of communication networks include a LAN, a WAN, the Internet, mobile telephone networks, Plain Old Telephone (POTS) networks, and wireless data networks (e.g., Wi-Fi and 4G/5G data networks). The term “transmission medium” shall be taken to include any intangible medium that is capable of storing, encoding or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Additional Notes

The above detailed description includes references to the accompanying drawings, which form a part of the detailed description. The drawings show, by way of illustration but not by way of limitation, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that after such a term (e.g., comprising, including, having) in a claim are still deemed to fall within the scope of that claim. Moreover, in the following claims, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Method examples (e.g., operations and functions) described herein can be machine or computer-implemented at least in part (e.g., implemented as software code or instructions). Some examples can include a computer-readable medium or machine-readable medium encoded with instructions operable to configure an electronic device to perform methods as described in the above examples. An implementation of such methods can include software code, such as microcode, assembly language code, a higher-level language code, or the like (e.g., “source code”). Such software code can include computer-readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the internet, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMs (e.g., CMOS RAM and the like), recordable/non-recordable media (e.g., read only memories (ROMs)), EPROMS, EEPROMS, or any type of media suitable for storing electronic instructions, and the like. Such computer-readable storage medium is coupled to a computer system bus to be accessible by the processor and other parts of the OIS.

In an embodiment, the computer-readable storage medium may have encoded a data structure for treatment planning, wherein the treatment plan may be adaptive. The data structure for the computer-readable storage medium may be at least one of a Digital Imaging and Communications in Medicine (DICOM) format, an extended DICOM format, an XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module can be implemented in software, hardware, or a combination thereof. The methods provided by various embodiments of the present disclosure, for example, can be implemented in software by using standard programming languages such as, for example, C, C++, Java, Python, and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface can be configured by providing configuration parameters and/or sending signals to prepare the communication interface to provide a data signal describing the software content. The communication interface can be accessed via one or more commands or signals sent to the communication interface.

The present disclosure also relates to a system for performing the operations herein. This system may be specially constructed for the required purposes, or it may comprise a general purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved and other advantageous results attained. Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

1.-30. (canceled)

31. A computing system for generating radiotherapy machine parameters used in a radiotherapy treatment plan, the computing system comprising:
one or more memory devices to store a three-dimensional set of image data corresponding to a subject of radiotherapy treatment, the image data indicating one or more target dose areas and one or more organs-at-risk areas in anatomy of the subject; and
one or more processors configured to perform operations that:
generate anatomy projection images from the image data, each anatomy projection image providing a view of the subject from a respective beam angle of the radiotherapy treatment;
use a trained neural network model to generate control point images based on the anatomy projection images, each of the control point images indicating an intensity and one or more apertures of a control point of the radiotherapy treatment to apply at a respective beam angle, wherein the neural network model is trained with corresponding pairs of training anatomy projection images and training control point images; and
generate a set of final control points for use in the radiotherapy treatment to control a radiotherapy treatment machine, based on optimization of the control points of the radiotherapy treatment indicated by the generated control point images;
wherein the neural network model is trained by a generative adversarial network (GAN) or a conditional generative adversarial network (cGAN), and wherein neural network parameter values learned by the neural network model are established from adversarial training in the GAN or cGAN.

32. The computing system of claim 31, wherein beam angles of the radiotherapy treatment correspond to gantry angles of the radiotherapy treatment machine.

33. The computing system of claim 32, wherein operations to obtain the three-dimensional set of image data corresponding to a subject includes operations to obtain image data for each gantry angle of the radiotherapy treatment machine, and wherein each generated anatomy projection image represents a view of the anatomy of the subject from a given gantry angle used to provide treatment with a given radiotherapy beam.

34. The computing system of claim 31, wherein the radiotherapy treatment comprises a volume modulated arc therapy (VMAT) radiotherapy performed by the radiotherapy treatment machine, wherein multiple radiotherapy beams are shaped to achieve a modulated dose for target areas, from among multiple beam angles, to deliver a prescribed radiation dose.

35. The computing system of claim 34, further comprising:
using fluence data to determine radiation doses in the radiotherapy treatment plan, wherein the trained neural network model is further configured to generate the control point images based on the fluence data;
wherein the fluence data is provided from fluence maps, wherein the neural network model is further trained with fluence maps corresponding to the training anatomy projection images and the training control point images; and
wherein the fluence maps are provided from use of a second trained neural network model configured to generate the fluence maps based on the anatomy projection images, each of the generated fluence maps indicating a fluence distribution of the radiotherapy treatment at a respective beam angle, wherein the second neural network model is trained with corresponding pairs of the anatomy projection images and fluence maps.

36. The computing system of claim 31, wherein each anatomy projection image is generated by forward projection of the three-dimensional set of image data at respective angles of multiple beam angles.

37. The computing system of claim 31, wherein training of the neural network model uses pairs of anatomy projection images and control point images for a plurality of human subjects, wherein each individual pair is provided from a same human subject, and wherein the neural network model is trained with operations that:
obtain multiple sets of training anatomy projection images, each set of the training anatomy projection images indicating one or more target dose areas and one or more organs-at-risk areas in the anatomy of a respective subject;
obtain multiple sets of training control point images corresponding to the training anatomy projection images, each set of the training control point images indicating a control point for at a respective beam angle of the radiotherapy machine used with radiotherapy treatment of the respective subject; and
train the neural network model based on the training anatomy projection images that correspond to the training control point images.

38. The computing system of claim 37, wherein the GAN or the cGAN comprises at least one generative model and at least one discriminative model, wherein the at least one generative model and the at least one discriminative model correspond to respective generative and discriminative convolutional neural networks.

39. A non-transitory computer-readable storage medium comprising computer-readable instructions for generating radiotherapy machine parameters used in a radiotherapy treatment plan, the instructions performing operations comprising:
obtaining a three-dimensional set of image data corresponding to a subject for radiotherapy treatment, the image data indicating one or more target dose areas and one or more organs-at-risk areas in anatomy of the subject;
generating anatomy projection images from the image data, each anatomy projection image providing a view of the subject from a respective beam angle of the radiotherapy treatment;
using a trained neural network model to generate control point images based on the anatomy projection images, each of the control point images indicating an intensity and one or more apertures of a control point of the radiotherapy treatment to apply at a respective beam angle, wherein the neural network model is trained with corresponding pairs of training anatomy projection images and training control point images; and
generating a set of final control points for use in the radiotherapy treatment to control a radiotherapy treatment machine, based on optimization of the control points of the radiotherapy treatment indicated by the generated control point images;
wherein the neural network model is trained by a generative adversarial network (GAN), and wherein neural network parameter values learned by the neural network model are established from adversarial training in the GAN or cGAN.

40. The computer-readable storage medium of claim 39, wherein beam angles of the radiotherapy treatment correspond to gantry angles of the radiotherapy treatment machine.

41. The computer-readable storage medium of claim 40, wherein operations to obtain the three-dimensional set of image data corresponding to a subject includes operations to obtain image data for each gantry angle of the radiotherapy treatment machine, and wherein each generated anatomy projection image represents a view of the anatomy of the subject from a given gantry angle used to provide treatment with a given radiotherapy beam.

42. The computer-readable storage medium of claim 39, wherein the radiotherapy treatment comprises a volume modulated arc therapy (VMAT) radiotherapy performed by the radiotherapy treatment machine, and wherein multiple radiotherapy beams are shaped to achieve a modulated dose for target areas, from among multiple beam angles, to deliver a prescribed radiation dose.

43. The computer-readable storage medium of claim 42, further comprising:
using fluence data to determine radiation doses in the radiotherapy treatment plan, wherein the trained neural network model is further configured to generate the control point images based on the fluence data;
wherein the fluence data is provided from fluence maps, wherein the neural network model is further trained with fluence maps corresponding to the training anatomy projection images and the training control point images; and
wherein the fluence maps are provided from use of a second trained neural network model configured to generate the fluence maps based on the anatomy projection images, each of the generated fluence maps indicating a fluence distribution of the radiotherapy treatment at a respective beam angle, wherein the second neural network model is trained with corresponding pairs of the anatomy projection images and fluence maps.

44. The computer-readable storage medium of claim 39, wherein each anatomy projection image is generated by forward projection of the three-dimensional set of image data at respective angles of multiple beam angles.

45. The computer-readable storage medium of claim 39, wherein training of the neural network model uses pairs of anatomy projection images and control point images for a plurality of human subjects, wherein each individual pair is provided from a same human subject, and wherein the neural network model is trained with operations comprising:
obtaining multiple sets of training anatomy projection images, each set of the training anatomy projection images indicating one or more target dose areas and one or more organs-at-risk areas in the anatomy of a respective subject;
obtaining multiple sets of training control point images corresponding to the training anatomy projection images, each set of the training control point images indicating a control point for at a respective beam angle of the radiotherapy machine used with radiotherapy treatment of the respective subject; and
training the neural network model based on the training anatomy projection images that correspond to the training control point images.

46. The computer-readable storage medium of claim 45, wherein the GAN comprises at least one generative model and at least one discriminative model, wherein the at least one generative model and the at least one discriminative model correspond to respective generative and discriminative convolutional neural networks.

47. A computer-implemented method for generating radiotherapy machine control parameters used in a radiotherapy treatment plan, the method comprising:
obtaining a three-dimensional set of image data corresponding to a subject for radiotherapy treatment, the image data indicating one or more target dose areas and one or more organs-at-risk areas in anatomy of the subject;
generating anatomy projection images from the image data, each anatomy projection image providing a view of the subject from a respective beam angle of the radiotherapy treatment;
using a trained neural network model to generate control point images based on the anatomy projection images, each of the control point images indicating an intensity and one or more apertures of a control point of the radiotherapy treatment to apply at a respective beam angle, wherein the neural network model is trained with corresponding pairs of training anatomy projection images and training control point images; and
generating a set of final control points for use in the radiotherapy treatment to control a radiotherapy treatment machine, based on optimization of the control points of the radiotherapy treatment indicated by the generated control point images;
wherein the neural network model is trained by a generative adversarial network (GAN), and wherein neural network parameter values learned by the neural network model are established from adversarial training in the GAN.

48. The method of claim 47, wherein beam angles of the radiotherapy treatment correspond to gantry angles of the radiotherapy treatment machine.

49. The method of claim 48, wherein obtaining the three-dimensional set of image data corresponding to a subject includes obtaining image data for each gantry angle of the radiotherapy treatment machine, and wherein each generated anatomy projection image represents a view of the anatomy of the subject from a given gantry angle used to provide treatment with a given radiotherapy beam.

50. The method of claim 47, wherein the radiotherapy treatment comprises a volume modulated arc therapy (VMAT) radiotherapy performed by the radiotherapy treatment machine, wherein multiple radiotherapy beams are shaped to achieve a modulated dose for target areas, from among multiple beam angles, to deliver a prescribed radiation dose.

51. The method of claim 50, further comprising:
using fluence data to determine radiation doses in the radiotherapy treatment plan, wherein the trained neural network model is further configured to generate the control point images based on the fluence data.

52. The method of claim 51, wherein the fluence data is provided from fluence maps, wherein the neural network model is further trained with fluence maps corresponding to the training anatomy projection images and the training control point images.

53. The method of claim 52, wherein the fluence maps are provided from use of a second trained neural network model configured to generate the fluence maps based on the anatomy projection images, each of the generated fluence maps indicating a fluence distribution of the radiotherapy treatment at a respective beam angle, wherein the second neural network model is trained with corresponding pairs of the anatomy projection images and fluence maps.

54. The method of claim 47, wherein each anatomy projection image is generated by forward projection of the three-dimensional set of image data at respective angles of multiple beam angles.

55. The method of claim 47, wherein training of the neural network model uses pairs of anatomy projection images and control point images for a plurality of human subjects, wherein each individual pair is provided from a same human subject, and wherein the neural network model is trained with operations comprising:
obtaining multiple sets of training anatomy projection images, each set of the training anatomy projection images indicating one or more target dose areas and one or more organs-at-risk areas in the anatomy of a respective subject;
obtaining multiple sets of training control point images corresponding to the training anatomy projection images, each set of the training control point images indicating a control point for at a respective beam angle of the radiotherapy machine used with radiotherapy treatment of the respective subject; and
training the neural network model based on the training anatomy projection images that correspond to the training control point images.

56. The method of claim 55, wherein the GAN comprises at least one generative model and at least one discriminative model, and wherein the at least one generative model and the at least one discriminative model correspond to respective generative and discriminative convolutional neural networks.

57. The method of claim 56, wherein the GAN comprises a conditional generative adversarial network (cGAN), wherein the training in the cGAN is conditioned by training images that represent conditions including one or more radiotherapy treatment target areas or one or more organs at risk areas.

58. The method of claim 47, wherein the optimization of the control points produces a pareto-optimal plan used in the radiotherapy treatment plan for the subject.

59. The method of claim 47, wherein the optimization of the control points comprises performing direct aperture optimization with aperture settings, wherein the set of final control points includes control points corresponding to each of multiple radiotherapy beams.

60. The method of claim 59, further comprising:
causing the radiotherapy treatment to be performed, using the set of final control points, wherein the set of final control points are used to control multi-leaf collimator (MLC) leaf positions of the radiotherapy treatment machine at a given gantry angle corresponding to a given beam angle.
